(19) United States
(12) Patent Application Publication
(10) Pub. No.: US 2024/0285769 A1
(43) Pub. Date: Aug. 29, 2024

(54) INJECTABLE, BIODEGRADABLE AND REMOVABLE POLYMER BASED DRUG SUSPENSION FOR ULTRA-LONG-ACTING DRUG DELIVERY

(71) Applicant: The University of North Carolina at Chapel Hill, Chapel Hill, NC (US)

(72) Inventors: Soumya Rahima Benhabbour, Chapel Hill, NC (US); Panita Maturavongsadit, Chapel Hill, NC (US); Roopali Shrivastava, Chapel Hill, NC (US); Isabella C. Young, Chapel Hill, NC (US)

(21) Appl. No.: 18/571,412

(22) PCT Filed: Jun. 30, 2022
(86) PCT No.: PCT/US2022/035713

§ 371 Date: Dec. 18, 2023

Related U.S. Application Data
(60) Provisional application No. 63/217,150, filed on Jun. 30, 2021.

(51) Int. Cl. A61K 47/22; A61K 9/00; A61K 47/20; A61K 47/34

(52) U.S. Cl. CPC A61K 47/22; A61K 9/0024; A61K 47/20; A61K 47/34

(57) ABSTRACT

Injectable, biodegradable and removable polymer based drug suspension for ultra-long-acting drug delivery are disclosed. Ultra-long-acting in-situ forming implant (ISFI) drug suspension delivery systems as multipurpose prevention technologies for a multitude of applications are also provided. Methods of use, including treatment of subjects, using the disclosed compositions and ISFIs are also provided.

### CROSS-REFERENCE TO RELATED APPLICATIONS

This application claims benefit of U.S. Provisional Patent Application Ser. No. 63/217,150, filed Jun. 30, 2021, herein incorporated by reference in its entirety.

### GRANT STATEMENT

This invention was made with government support under Grant No. AI162246 and AI131430 awarded by the National Institutes of Health. The government has certain rights in the invention.

### TECHNICAL FIELD

The presently disclosed subject matter is directed to injectable, biodegradable and removable polymer based drug suspension for ultra-long-acting drug delivery. More particularly, the presently disclosed subject matter is directed to ultra-long-acting in-situ forming implant (ISFI) drug suspension delivery systems as multipurpose prevention technologies for a multitude of application.

### BACKGROUND

Multipurpose prevention technologies (MPT) for protection of women against sexually transmitted pathogens and prevention of pregnancy are in a phase of accelerated encouragement and development, with multiple drugs and delivery systems. Long-acting (LA) pre-exposure prophylaxis (PrEP) formulations that provide sustained drug release over weeks or months can potentially enhance compliance to prophylactic therapies and reduce the incidence of new HIV infections and unplanned pregnancy. Moreover, injectable contraceptive use is highly acceptable in Africa and has increased substantially over the past few decades. In many countries in Africa where HIV incidence is high, the intramuscular injectable progestin depot medroxyprogesterone acetate (DMPA-IM) is the predominant contraceptive used. Recent results from the ECHO study showed no difference in HIV risk between DMPA-IM, Nexplanon and copper IUD, and all methods were safe and highly effective (Hapgood 2020, Tepper, Curtis et al. 2020). Currently, there are no LA injectable MPT formulations in development mainly because of limitations of current LA injectable formulations utilizing nanoparticle suspensions like cabotegravir and rilpivirine injectable formulations (Cabenuva®, Apretude®). These limitations include inability to combine two drugs into one formulation and of importance, once administered, nanoparticle formulated LA injectable drugs cannot be removed in the event of breakthrough infection, toxicity, allergic response, or pregnancy. To address these limitations we set out to develop and implement a novel, ultra-LA In-Situ Forming Implant (ISFI) drug suspension delivery system as a MPT for a multitude of applications, including for example prevention of HIV and unplanned pregnancy. In some embodiments the disclosed ultra-LA ISFI formulations can 1) be simple to prepare, 2) can incorporate an antiretroviral and a contraceptive drug with an initial targeting of greater than or equal to about 6 months of sustained release, and 3) can be removed in case of toxicity, breakthrough infection, or allergic response.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided are stable polymer-based injectable suspensions, the stable polymer-based injectable suspension comprising a polymer, optionally a combination of polymers and/or a combination of a polymer(s) and an additive(s)/stabilizer(s); a solvent, optionally a combination of solvents; and a drug, optionally a combination of one or more drugs, in a suspension. In some embodiments, the drug is in the suspension at a concentration beyond a saturation concentration in a placebo formulation, wherein the placebo formulation comprises the polymer and the solvent. In some embodiments, the stable polymer-based injectable suspension is injectable into a subject, wherein the stable polymer-based injectable suspension forms a biodegradable in-situ forming implant (ISFI) when injected into a subject. In some embodiments, the stable polymer-based injectable suspension comprises one or more hydrophobic molecules or components, or a combination of hydrophobic and hydrophilic molecules. In some embodiments, the ratio of polymer:solvent in the suspension ranges from about 1:1 to about 1:6, optionally, wherein the ratio of polymer:solvent is about 1:1, about 1:1.25, about 1.1.5, about 1:2, about 1:2.5, about 1:3, about 1:3.5, about 1:4, about 1:4.5, about 1:5, about 1:5.5, or about 1:6. In some embodiments, the ratio of polymer:drug in the suspension ranges from about 1:1, about 1:1.5, about 1:2, about 1:2.5, about 1:3, about 1:3.5, about 1:4, and 1:4.5, about 1:5, about 1:5.5, or about 1:6. In some embodiments, the polymer is a biodegradable polymer, optionally wherein the polymer is selected from the group consisting of polyesters e.g. poly-lactic-co-glycolic acid (PLGA), poly-lactic acid (PLA), polyglycolic acid (PGA); polycaprolactone (PCL); Poly hydroxyl butyrate (PUB); polyethylene glycol (PEG); sucrose acetate isobutyrate (SAIB); polyamides; polyanhydrides; polyphosphazenes; polyacrylates; polyorthoesters; polyalkylcyanoacrylates; polyurethanes; poly(ester amides); poly(ester urea); poly(phosphoesters); polysaccharides; hyaluronic acid; chitosan; alginate; collagen; arginine; albumin; dextran; gelatin; agarose; carrageenan; biomimetic and bio-inspired polymers or combinations thereof. In some embodiments, the molecular weight (MW) of the polymer ranges from about 5 kDa to about 100 kDa, optionally wherein the MW of the polymer is about 10 kDa or about 55 kDa. In some embodiments, the solvent is a water-miscible biocompatible solvent, optionally wherein the solvent is selected from the group consisting of N-methyl-2-pyrrolidone (NMP), dimethyl sulfoxide (DMSO), benzyl benzoate (BB), triacetin (TA) and combinations thereof. In some embodiments, the solvent comprises a mixture of NNP and DMSO at a ratio of about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, or about 1:9 v/v. In some embodiments, the stable polymer-based injectable suspension comprises one or more drugs, biologics, active agents, contrast agents and/or therapeutic compounds. In some embodiments, the drug comprises one or more drugs, optionally wherein the drug comprises biologics, active agents, contrast agents and/or therapeutic compounds, optionally wherein the drug is an antiretroviral drug, e.g. Cabotegravir (CAB), Dolutegravir (DTG), Doravirine (DOR), lamuvidine (3TC), and Islatravir (EFdA), emtricitabine (FTC), tenofovir disoproxil fumarate (TDF) or tenofovir alafenamide (TAF), optionally wherein the drug is a chemotherapeutic drug or agent, e.g. paclitaxel (PTX) and gemcitabine (Gem). In some embodiments, the contrast agent is a radiopaque, e.g. barium sulphate; optionally wherein the contrast agent is radioactive, e.g. iodide, gadolinium; optionally wherein the contrast agent is a fluorophore, e.g. fluorescein, indocyanine green, green fluorescent protein (GFP), m-cherry, optionally wherein the contrast agent is bioluminescent agent, e.g. luciferin. In some embodiments, the suspension comprises the polymer PLGA (e.g. MW 10 or 27 kDa) and a NMP and DMSO solvent mixture (e.g. 1:1 v/v) at a ratio of about 1:2 to about 1:6, with a drug (e.g. CAB) at a concentration of about 200 mg/g to about 600 mg/g. In some embodiments, the suspension has a high drug loading capacity, optionally up to about 600 mg/mL. In some embodiments, the suspension is configured to accommodate one or more drugs at concentrations ranging from about 5 wt % to about 85 wt % which is translatable to a human dose required to achieve therapeutic effect. In some embodiments, the suspension and/or a biodegradable ISFI formed therefrom is configured to provide ultra-long-acting drug release of about 90 days or more. In some embodiments, the suspension and/or a biodegradable ISFI formed therefrom is configured to provide drug release based on diffusion and biodegradation. In some embodiments, the suspension and/or a biodegradable ISFI formed therefrom is configured to provide a low 24 hour drug burst release rate (e.g. less than about 5% of the total drug load in the suspension and/or biodegradable ISFI, optionally less than about 1% of the total drug load in the suspension and/or biodegradable ISFI). In some embodiments, the suspension and/or a biodegradable ISFI formed therefrom is removable from a subject after injection. In some embodiments, the polymer type, polymer MW, polymer architecture, solvent type, rate-controlling additives (e.g. pluronics, SAIB, Trehalose), stabilizers (e.g. Tween 20, Tween 80, polysorbate 20, mannitol, polyethylene glycol), ratio of polymer:drug, ratio of polymer:solvent, ratio of solvent:drug, ratio of polymer:solvent:drug, ratio of polymer:additives, and/or ratio of polymer:stabilizers is adjustable to provide a stable suspension and/or a biodegradable ISFI. In some embodiments, the suspension and/or a biodegradable ISFI formed therefrom is configured to provide co-delivery of multiple drugs in a single stable suspension formulation with superior control over drug loading and release kinetics.

In some embodiments, provided herein are biodegradable in-situ forming implants (ISFI) made from a stable polymer-based injectable suspension as disclosed herein. In some embodiments, provided are biodegradable in-situ forming implant (ISFI) made from a stable polymer-based injectable suspension, wherein the stable polymer-based injectable suspension comprises: a polymer, optionally a combination of polymers and/or a combination of a polymer(s) and an additive(s)/stabilizer(s); a solvent, optionally a combination of solvents; and a drug, optionally a combination of one or more drugs, in a suspension. In some embodiments, the drug is in the suspension at a concentration beyond a saturation concentration in a placebo formulation, wherein the placebo formulation comprises the polymer and the solvent. In some embodiments, the stable polymer-based injectable suspension is injectable into a subject, wherein the stable polymer-based injectable suspension forms a biodegradable in-situ forming implant (ISFI) when injected into a subject. In some embodiments, the stable polymer-based injectable suspension comprises one or more hydrophobic molecules or components, or a combination of hydrophobic and hydrophilic molecules. In some embodiments, the ratio of polymer:solvent in the suspension ranges from about 1:1 to about 1:6, optionally, wherein the ratio of polymer:solvent is about 1:1, about 1:1.25, about 1.1.5, about 1:2, about 1:2.5, about 1:3, about 1:3.5, about 1:4, about 1:4.5, about 1:5, about 1:5.5, or about 1:6. In some embodiments, the ratio of polymer:drug in the suspension ranges from about 1:1, about 1:1.5, about 1:2, about 1:2.5, about 1:3, about 1:3.5, about 1:4, and 1:4.5, about 1:5, about 1:5.5, or about 1:6. In some embodiments, the polymer is a biodegradable polymer, optionally wherein the polymer is selected from the group consisting of polyesters e.g. poly-lactic-co-glycolic acid (PLGA), poly-lactic acid (PLA), polyglycolic acid (PGA); polycaprolactone (PCL); Poly hydroxyl butyrate (PHB); polyethylene glycol (PEG); sucrose acetate isobutyrate (SAIB); polyamides; polyanhydrides; polyphosphazenes; polyacrylates; polyorthoesters; polyalkylcyanoacrylates; polyurethanes; poly(ester amides); poly(ester urea); poly(phosphoesters); polysaccharides; hyaluronic acid; chitosan; alginate; collagen; arginine; albumin; dextran; gelatin; agarose; carrageenan; biomimetic and bio-inspired polymers or combinations thereof. In some embodiments, the molecular weight (MW) of the polymer ranges from about 5 kDa to about 100 kDa, optionally wherein the MW of the polymer is about 10 kDa or about 55 kDa. In some embodiments, the solvent is a water-miscible biocompatible solvent, optionally wherein the solvent is selected from the group consisting of N-methyl-2-pyrrolidone (NMP), dimethyl sulfoxide (DMSO), benzyl benzoate (BB), triacetin (TA) and combinations thereof. In some embodiments, the solvent comprises a mixture of NMP and DMSO at a ratio of about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, or about 1:9 v/v. In some embodiments, the stable polymer-based injectable suspension comprises one or more drugs, biologics, active agents, contrast agents and/or therapeutic compounds. In some embodiments, the drug comprises one or more drugs, optionally wherein the drug comprises biologics, active agents, contrast agents and/or therapeutic compounds, optionally wherein the drug is an antiretroviral drug, e.g. Cabotegravir (CAB), Dolutegravir (DTG), Doravirine (DOR), lamuvidine (3TC), and Islatravir (EFdA), emtricitabine (FTC), tenofovir disoproxil fumarate (TDF) or tenofovir alafenamide (TAF), optionally wherein the drug is a chemotherapeutic drug or agent, e.g. paclitaxel (PTX) and gemcitabine (GemIn some embodiments, the contrast agent is a radiopaque, e.g. barium sulphate; optionally wherein the contrast agent is radioactive, e.g. iodide, gadolinium; optionally wherein the contrast agent is a fluorophore, e.g. fluorescein, indocyanine green, green fluorescent protein (GFP), m-cherry: optionally wherein the contrast agent is bioluminescent agent, e.g. luciferin. In some embodiments, the suspension comprises the polymer PLGA (e.g. MW 10 or 27 kDa) and a NMP and DMSO solvent mixture (e.g. 1:1 v/v) at a ratio of about 1:2 to about 1:6, with a drug (e.g. CAB) at a concentration of about 200 mg/g to about 600 mg/g. In some embodiments, the suspension has a high drug loading capacity, optionally up to about 600 mg/mL. In some embodiments, the suspension is configured to accommodate one or more drugs at concentrations ranging from about 5 wt % to about 85 wt % which is translatable to a human dose required to achieve therapeutic effect. In some embodiments, the suspension and/or a biodegradable ISFI formed therefrom is configured to provide ultra-long-acting drug release of about 90 days or more. In some embodiments, the suspension and/or a biodegradable ISFI formed therefrom is configured to provide drug release based on diffusion and biodegradation. In some embodiments, the suspension and/or a biodegradable ISFI formed therefrom is configured to provide a low 24 hour drug burst release rate (e.g. less than about 5% of the total drug load in the suspension and/or biodegradable ISFI, optionally less than about 1% of the total drug load in the suspension and/or biodegradable ISFI). In some embodiments, the suspension and/or a biodegradable ISFI formed therefrom is removable from a subject after injection. In some embodiments, the polymer type, polymer MW, polymer architecture, solvent type, rate-controlling additives (e.g. pluronics, SAIB, Trehalose), stabilizers (e.g. Tween 20, Tween 80, polysorbate 20, mannitol, polyethylene glycol), ratio of polymer:drug, ratio of polymer:solvent, ratio of solvent:drug, ratio of polymer:solvent:drug, ratio of polymer:additives, and/or ratio of polymer:stabilizers is adjustable to provide a stable suspension and/or a biodegradable ISFI. In some embodiments, the suspension and/or a biodegradable ISFI formed therefrom is configured to provide co-delivery of multiple drugs in a single stable suspension formulation with superior control over drug loading and release kinetics. In some embodiments, the biodegradable ISFI configured to be removable from a subject after implantation to terminate the treatment if required. In some embodiments, the biodegradable ISFI is syringeable and/or injectable. In some embodiments, the biodegradable ISFI is configured to accommodate one or more drugs, biologics, active agents, contrast agent and/or therapeutic compounds. In some embodiments, the biodegradable ISFI has a high drug loading capacity, optionally up to about 600 mg/mL. In some embodiments, the biodegradable ISFI is configured to provide ultra-long-acting drug release of about 90 days or more.

Provided in some embodiments are methods of administering a drug, active agent, contrast agent and/or therapeutic compound to a subject, the method comprising providing a stable polymer-based injectable suspension and/or biodegradable ISFI of any of claims 1 to 50, and administering the same to a subject in need of receiving a drug, biologic, active agent, contrast agent and/or therapeutic compound. In some embodiments the methods further comprise loading the stable polymer-based injectable suspension and/or biodegradable ISFI with one or more drugs, active agents, contrast agent and/or therapeutic compounds prior to administration to the subject, optionally wherein the one or more drugs, active agents and/or therapeutic compounds comprises an antiviral, antibacterial, antifungal, contraceptive, prophylactic, anti-inflammatory, anticancer, analgesic, hormone, steroid, opioid and combinations thereof. In some embodiments, the stable polymer-based injectable suspension and/or biodegradable ISFI is administered via injection. In some embodiments, the stable polymer-based injectable suspension and/or biodegradable ISFI is configured to be removable from the subject if required to terminate the treatment. In some embodiments, the subject is a human subject.

Provided in some embodiments are methods of treating a subject, the methods comprising administering to a subject in need of treatment a stable polymer-based injectable suspension and/or biodegradable ISFI of any of claims 1 to 50, wherein the stable polymer-based injectable suspension and/or biodegradable ISFI comprises a drug, biologic, active agent, contrast agent and/or therapeutic compound. In some embodiments, the drug, biologic, active agent, contrast agent and/or therapeutic compound comprises an antiviral, antibacterial, antifungal, contraceptive, prophylactic, anti-inflammatory, anticancer, analgesic, hormone, steroid, opioid and combinations thereof. In some embodiments, the stable polymer-based injectable suspension and/or biodegradable ISFI is administered via injection. In some embodiments, the subject is a human subject.

These and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1. Cabotegravir formulated in 1:2 PLGA/(NMP:DMSO) ISFI as a solution (CAB 100 mg/mL) or as a suspension (CAB ≥200 mg/mL).

FIGS. 2A and 2B. Ternary phase diagrams of: FIG. 2A, CAB ISFI formulations and FIG. 2B DTG ISFI formulations that are formulated as solutions (circles), suspensions (squares), and proposed (triangles).

FIG. 3. ISFI microstructure of single-drug ISFIs and DTG MPT ISFIs. SEM images of DTG, ENG, MPA, DTG/ENG, and DTG/MPA ISFIs (by column left to right, respectively) 3 days post-incubation in vitro. Each column within the matrix represents increasing magnification (70×, 100×, and 200×, top to bottom, respectively). This increase in pores is likely driven by MPA due to its basic pKa increasing the rate of PLGA degradation via hydrolysis (Holy, Dang et al. 1999, Makadia and Siegel 2011). Alternatively, ENG ISFIs elicit a dense and compact microstructure which may be causing ENG to be trapped inside the depot until bulk degradation of the polymer begins after approximately 30 days. This phenomenon is not observed when ENG is co-formulated with DTG as DTG/ENG ISFIs demonstrate a more porous microstructure promoting the release of both drugs.

FIG. 4. ISFI microstructure of single-drug ISFIs and MPT ISFIs. SEM images of CAB, ENG, MPA, CAB/MPA, and CAB/ENG ISFIs (by column left to right, respectively) 3 days post-incubation in vitro. Each column within the matrix represents increasing magnification (70×, 100×, and 200×, top to bottom, respectively). Scale bars represent 100 μm.

FIGS. 5A to 5D. CAB ISFI cumulative in vitro release kinetics. (FIG. 5A) Cumulative release of CAB ISFI formulations. (FIG. 5B) Effect of drug loading on cumulative CAB release. (FIG. 5C) Effect of PLGA molecular weight on cumulative CAB release. (FIG. 5D) Summary table of release kinetics for CAB ISFI formulations. Solvent=1:1 (w/w) NMP:DMSO. All in vitro release studies were done in triplicate in phosphate buffer saline (PBS, pH 7.4 with 2% solutol) at 37° C. Error bars represent standard deviation for n=3 samples.

FIGS. 6A and 6B. Effect of formulation composition on CAB release kinetics, including percent cumulative release (FIG. 6A) and cumulative amount released (FIG. 6B).

FIGS. 7A and 7B. In vitro release of DOR from ISFI suspension in PBS at 37° C. Samples were collected at predetermined timepoints and analyzed by HPLC to quantify DOR concentration (FIG. 7A) and determine release kinetics over time (FIG. 7B).

FIGS. 8A to 8E. In vivo safety evaluation and in vivo drug release of CAB ISFIs in BALB/c mice. (FIG. 8A) Local inflammation of excised depots and surrounding subcutaneous tissues collected at day 3, 7, and 30 post-injection (n=3/timepoint) and stained with H&E. Asterisks indicate CAB implant. Arrows indicate infiltrated immune cells and areas of inflammation. All scale bars represent 1 mm. (FIG. 8B) Inflammatory score of subcutaneous tissue surrounding the depot evaluated using a light microscope and scored blindly by a certified pathologist. Black bars represent the median inflammation score at each timepoint (n=3 per timepoint). Inflammation scoring: 0: inflammatory cells present within expected limits; 1: minimal inflammation, few increased, scattered immune cells present; 2: mild inflammation, small clusters of immune cells to thin or localized tracks of inflammation or mild increase of the number of cells diffusely surrounding the depot; 3, moderate, thicker or multiple tracks of inflammation or moderate numbers of cells diffusely surrounding the depot; 4, severe, coalescing tracks of inflammation large enough to replace normal tissue architecture or severe numbers of cells diffusely surrounding the depot; 5, marked, inflammation present that is replacing expansive areas of normal tissue architecture. (FIG. 8C) Concentration of TNF-α (pg/mL) in plasma quantified by ELISA at day 3 (n=3), 7 (n=3), and 30 (n=5) post-injection. (FIG. 8D) Concentration of IL-6 (pg/mL) in plasma quantified by ELISA at day 3 (n=3), 7 (n=3), and 30 (n=5) post-injection. (FIG. 8E) CAB concentration (average±standard deviation) in plasma (1215 mg/kg) for 90 days (n=6-12 per timepoint). 1× and 4×PA-IC90 values are indicated with dotted lines for CAB (166 ng/mL and 664 ng/mL, respectively).

FIGS. 9A to 9D. Cabotegravir concentrations in plasma and tissues in rhesus macaques treated with two CAB ISFIs. FIG. 9A. Longitudinal assessment of CAB concentrations in plasma. The 2 CAB ISFIs were removed from macaques RH-1097 and 1093 (blue and purple solid circles) at week 12 and one of the two CAB ISFIs was removed from macaque RH-42012 at week 14 (green solid circle) FIG. 9B. Asterisk (*) indicates animals with ISFIs removed at weeks 12-14. Data after implant removal is not included in the calculation of medians. FIG. 9C. Median concentrations of CAB in plasma, rectal tissues, and vaginal tissues at 4, 8, and 12 weeks. Error bars represent the range. FIG. 9D. Ratio of CAB concentrations in vaginal tissues (VT) and rectal tissues (RT) relative to plasma.

FIGS. 10A to 10D. Efficacy of CAB ISFI against rectal SHIV infection in rhesus macaques. In FIGS. 10A and 10B short-term protection by CAB ISFIs. Two CAB treated (RH-1093 and RH-1097) and one untreated macaque (RH-1092) were exposed to SHIV between weeks 4 and 8 post implantation (FIG. 10A). Each animal received two weekly rectal SHIV challenges or a total of 8 exposures (denoted by the arrows). ISFIs in treated animals were removed at week 12 (blue and purple solid circles) and animals were monitored weekly for SHIV infection by real-time PCR (FIG. 10B) In FIGS. 10C and 10D long-term protection by CAB ISFI. Two CAB treated (RH-1048 and RH-1080) and one untreated macaque (RH-1084) were exposed to SHIV between weeks 12 and 16 post implantation (FIG. 10C). Each animal received two weekly rectal SHIV challenges or 8 exposures (denoted by the arrows). Animal RH-1048 received 6 additional SHIV challenges between weeks 24 and 27. All animals were monitored weekly for SHIV infection by real-time PCR (FIG. 10D).

FIGS. 11A and 11B. Safety and tolerability of CAB ISFI implants in rhesus macaques. FIG. 11A. Heatmap of local skin reactions at the implant site. Local skin reactions were scored using a Draize scale (0-none to 4-severe). FIG. 11B. Histopathology of skin collected at the implantation sites from animals RH-1093, RH-1097, RH-42012 and untreated macaque was used a control. The full thickness skin punch biopsy shows no evidence of inflammation, infection, or foreign (implant) material present (left: H&E, original magnification ×2). The skins show no to minimal scattered interstitial and perivascular lymphoplasmacytic infiltrates (arrow) (right: H&E, original magnification ×40×).

FIGS. 12A to 12E. CAB ISFI biodegradation and residual drug quantification in BALB/c mice and rhesus macaques. (FIG. 12A) Image of CAB ISFIs retrieved from BALB/c mice 30-, 60-, and 90-days post-injection. (FIG. 12B) CAB ISFI masses 90 days post-injection in mice (n=3) compared to initial ISFI mass (day 0) from a 50 μL injection volume (60.75 mg). Day 0 masses were calculated based on a 50 μL injection volume and using the density of the formulation (1.215 g/mL) to determine approximate mass at injection. (FIG. 12C) PLGA degradation in CAB ISFIs 90 days post-injection in mice (n=2 per group) compared to neat PLGA (10 kDa). (FIG. 12D) Quantification of residual CAB in ISFIs 90 days post-injection in mice (n=3) compared to initial dose (day 0, about 24.3 mg in 50 μL injection). (FIG. 12E) Residual drug quantification and estimated release per day of implants (injected in the left and right upper back location) retrieved from three rhesus macaques 84- and 98-days post-injection.

FIGS. 13A and 13B. (FIG. 13A) CAB ISFI input rate is estimated by multiplying observed plasma concentrations with each respective animals' clearance as determined by their extravascular PK profile. Dashed and dotted reference lines denote input rates of 3 and 0.75 mg/day which are predicted to achieve plasma concentrations in humans above the 4×PA-IC90 and 1×PA-IC90, respectively. (FIG. 13B) Median (IQR) CAB ISFI input rates observed in this study (green line) are overlaid on median rates projected for a 3 mL injection volume (assuming input rate increases proportionally with volume; purple line) and on those estimated for 9 reference NHP dosed with 50 mg/kg intramuscular CAB LA at 7 and 1 days prior to PK sampling (22).

FIGS. 14A to 14D. In vitro release kinetics of CAB MPT ISFIs. Cumulative in vitro release of single-drug and MPT ISFIs over 90 days. (FIG. 14A) CAB/MPA ISFI and single-drug ISFI release. (FIG. 14B) CAB/ENG ISFI and single-drug ISFI release. (FIG. 14C) and (FIG. 14D) Summary table of release kinetics for CAB/MPA and CAB/ENG, respectively. Errors bars and standard deviations of n=3 samples.

FIGS. 15A to 15I. In vivo PK in BALB/c mice of CAB ISFIs (500 mg/mL CAB) to assess ISFI removability (FIGS. 15A-D), polymer degradation assessed via depot mass change overtime (FIG. 15E), residual CAB extracted from excised implants at various timepoints (FIG. 15F), polymer degradation assessed via decrease in molecular weight overtime (FIG. 15G), residual CAB quantified by LC-MS/MS analysis (FIG. 15H), and PLGA MW quantified by GPC analysis (FIG. 15I).

FIGS. 16A to 16G. Pharmacokinetic analysis of CAB ISFIs in plasma (FIG. 16A) and post ISFI removal (FIGS. 16B-D) and in tissues (FIGS. 16E-G).

FIGS. 17A to 17D. In vivo safety evaluation of MPT ISFIs. (FIG. 17A) Local inflammation of excised depot and surrounding subcutaneous tissue collected at day 3, 7, 30, and 90 post-injection (n=3/timepoint per group) and stained with H&E. Asterisk indicates ISFI depot. Arrows indicate infiltrated immune cells and areas of inflammation. All scale bars represent 1 mm. Zoomed in image of CAB/ENG ISFI at day 3 represents CAB crystals in depot (scale bar=100 μm). (FIG. 17B) Inflammatory score of subcutaneous tissue surrounding the depot evaluated using a light microscope and scored blindly by a certified pathologist. Black bars represent the median inflammation score of each group at each timepoint (n=3 per timepoint per group). (FIG. 17C) Concentration of TNF-α (pg/mL) in plasma quantified by ELISA at day 3 (n=3/group), 7 (n=3/group), 30 (n=6/group), 60 (n=3/group), and 90 post-injection (n=6/group). (FIG. 17D) Concentration of IL-6 (pg/mL) in plasma quantified by ELISA at day 3 (n=3/group), 7 (n=3/group), 30 (n=6/group), 60 (n=3/group), and 90 post-injection (n=6/group).

FIGS. 18A to 18E. Drug concentrations in plasma of MPT ISFI formulations. Plasma concentrations of (FIG. 18A) DTG (278 mg/kg, 50 μL injection) when co-formulated with MPA or ENG (n=6-12/timepoint per group), (FIG. 18B) CAB (1215 mg/kg, 50 μL injection) when co-formulated with MPA or ENG (n=6-12/timepoint per group), (FIG. 18C) MPA (70-76 mg/kg, 50 μL injection) when co-formulated with DTG or CAB (n=6-12/timepoint per group), and (FIG. 18D) ENG (70-151 mg/kg, 50 μL injection) when co-formulated with DTG or CAB (n=6-12/timepoint per group). 1× and 4×PA-IC90 values are indicated with dotted lines for DTG (64 ng/mL and 256 ng/mL) and CAB (166 ng/mL and 664 ng/mL). Plasma samples of individual mice are shown in Figure S9. (FIG. 18E) R² values for mathematical models of zero-order, first-order, and diffusion-controlled (Higuchi) release.

FIGS. 19A to 19D. Removal of MPT ISFIs and quantification of residual drug. (FIG. 19A) Image of MPT ISFI depots retrieved from mice 30-, 60-, and 90-days post-injection. (FIG. 19B) MPT ISFI masses 90 days post-injection in mice (n=3) compared to initial ISFI mass (day 0) from a 50 μL injection volume. Day 0 masses were calculated based on a 50 μL injection volume and using the density of the formulation (DTG MPT ISFI=1.113 g/mL and CAB MPT ISFIs=1.215 g/mL) to determine approximate mass at injection. (FIG. 19C) Quantification of residual DTG, ENG, and MPA in MPT ISFIs 90 days post-injection (n=3 per group) compared to initial dose (day 0) (initial doses: DTG about 5.5 mg; ENG about 1.4 mg or about 3.04 mg when formulated with DTG or CAB, respectively; MPA about 1.4 mg or about 1.5 mg when formulated with DTG or CAB, respectively). (FIG. 19D) Quantification of residual CAB in MPT ISFIs 90 days post-injection (n=3 per group) compared to initial dose (day 0) (initial dose: CAB about 24.3 mg).

FIGS. 20A to 20C. In vivo PLGA degradation of MPT ISFIs. (FIG. 20A) PLGA MW (weight average) of DTG/ENG and DTG/MPA ISFIs 90 days post-injection (n=2 per group) compared to neat PLGA (27 kDa). (FIG. 20B) PLGA MW (weight average) of CAB/ENG and CAB/MPA ISFIs 90 days post-injection (n=2 per group) compared to neat PLGA (10 kDa). (FIG. 20C) Summary table of PLGA MW decrease in MPT ISFIs.

FIGS. 21A to 21C. In vitro release studies of paclitaxel (PTX) ISFI formulations (FIG. 21C). Effects of drug loading and PLGA type (LA/GA ratio) on PTX release kinetics (FIGS. 21A and 21B).

FIGS. 22A to 22B. In vitro release studies of gemcitabine (Gem) ISFI formulations (FIG. 22B). Effects of PLGA/solvent ratio, rate controlling additives (Pluronic 61) and co-solvent (BB) on Gem release kinetics (FIG. 22A).

FIG. 23. KPC tumor growth of nude mice treated with SOC therapy (Abraxane+Gemzar), PTX ISFI, PTX ISFI+FUS (FUS=Focused Ultrasound), and Sham (no treatment).

FIG. 24. Images from in vivo X-ray imaging studies of CAB+BaSO₄ ISFI.

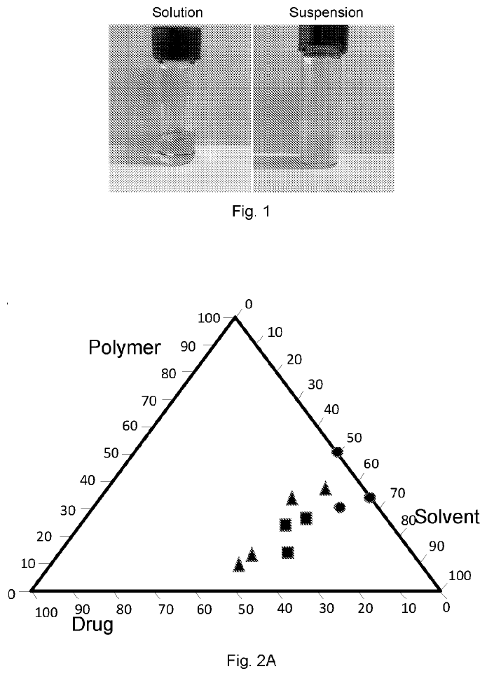

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the terms “treating,” “treatment”, and “to treat” are used to indicate the production of beneficial or desired results, such as to alleviate symptoms, or eliminate the causation of a disease or disorder either on a temporary or a permanent basis, slow the appearance of symptoms and/or progression of the disorder, or prevent progression of disease. The term “treat” or “treatment” refer to both therapeutic treatment and prophylactic or preventative measures, wherein the object is to prevent or slow down the development or spread of disease or symptoms. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total). “Treatment” can also refer to prolonging survival as compared to expected survival if not receiving treatment.

The term “subject”, “individual”, and “patient” are used interchangeably herein, and refer to an animal, especially a mammal, for example a human, to whom treatment, with a composition as described herein, is provided. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears.

The terms “long-acting”, “ultra-long-acting”, “sustained release”, “delayed release” and the like are used herein to refer to drug release over an extended period of time, including for example about 90 days or more, optionally about 30 days or more, about 60 days or more, about 90 days or more, about 120 days or more, about 150 days or more, or about 1, 2, 3, 4, 5, 6, 7, 8, 9, 10, 11, 12 months or more.

### II. Polymer-Based Injectable Suspension for Biodegradable and Removable In-Situ Forming Implants (ISFI)

Provided herein in some embodiments is a polymer-based injectable suspension that 1) when injected forms a biodegradable in-situ forming implant (ISFI); 2) can accommodate high drug payloads (up to about 50% w/w, optionally about 10% to about 70%, optionally about 20% to about 60%, optionally about 30% to about 50%, optionally about 10%, 20%, 30%, 40%, 50%, 60% or more); 3) can accommodate a plurality of drugs, e.g. active pharmaceutical agents (APIs), therapeutics, pharmaceutical compounds, etc., including for example two or more drugs in a single injection; 4) can be safely removed if required to terminate the treatment; and 5) allows ultra-long-acting delivery of drugs for an extended period of time, including several months (e.g. up to about 12 months or more).

During development of the disclosed polymer-based injectable suspensions it was desirable to develop compositions that can 1) accommodate one or more drugs, including for example antiretroviral drugs, at concentrations translatable to a human dose, 2) provide ultra-long-acting drug release of about 90 days or more, and 3) be removed from the body (in the case of an adverse/allergic event or pregnancy). Surprisingly, the results as disclosed herein showed that this type of stable suspension formulation can be achieved with hydrophobic molecules (e.g. dolutegravir, cabotegravir) and when these molecules are formulated at concentrations beyond their saturation concentration in the placebo ISFI formulation.

Further aspects and characteristics of some embodiments of the polymer-based injectable suspensions disclosed herein include the following:

1) Polymer based injectable suspension can in some embodiments provide superior drug loading (up to about 600 mg/mL) and superior control over drug release and duration compared to conventional in-situ forming implant solutions, injectable drug nanosuspensions (e.g. Elan technology), and injectable nanoparticle formulations.

2) Formulation composition (polymer type, polymer MW, polymer architecture, solvent type, ratio of polymer:solvent, ratio of polymer:drug, ratio of drug:solvent, additives and stabilizers) can be optimized to form a stable drug suspension and control drug loading and release characteristics specifically applicable to single or combination therapies will be significantly more effective compared to other LA injectable formulations currently being developed.

3) Formulation parameters can in some embodiments provide co-delivery of multiple drugs in a single stable suspension formulation with superior control over drug loading and release kinetics.

Globally, 38 million people are currently living with HIV and 36 million people have died from AIDS-related illnesses since the start of the epidemic (UNAIDS). Pre-exposure prophylaxis (PrEP) with daily oral regimens containing emtricitabine (FTC) in combination with tenofovir disoproxil fumarate (TDF) or tenofovir alafenamide (TAF) have been highly effective in preventing HIV acquisition when taken as prescribed (Baeten, Donnell et al. 2012, Thigpen, Kebaabetswe et al. 2012). However, low levels of adherence particularly among younger women have limited the effectiveness of oral PrEP and its public health impact. To this end, the pipeline for HIV prevention options is moving to develop long-acting (LA) PrEP products that do not require frequent dosing and may overcome some of the adherence challenges associated with daily oral PrEP.

An injectable long-acting formulation of the integrase inhibitor cabotegravir (CAB LA) was approved in late 2021 by the FDA for PrEP in men and women (FDA 2021). The approval of CAB LA followed results from the HPTN 083 and 084 trials showing that CAB LA was safe and more effective than daily oral FTC/TDF, likely reflecting the adherence advantage of long-acting PrEP (Landovitz 2021, Landovitz, Donnell et al. 2021, Marzinke, Grinsztejn et al. 2021). The studies also defined the plasma CAB concentrations needed for protection to be four times above the protein-adjusted 90% inhibitory concentration (4×PA-IC₉₀, 664 ng/mL (Trezza, Ford et al. 2015)). CAB LA is administered in 3 mL intramuscular injections twice monthly initially and bi-monthly thereafter. Efforts are now shifting to the development of ultra-long-acting CAB formulations that sustain protective plasma drug levels through extended dosing intervals such as every six months or longer. Such formulations facilitate large-scale implementation and maximize cost-effectiveness and public health benefit in both resource-poor and -rich countries. Because existing CAB LA is not removable it results in a long pharmacologic tail after discontinuation requiring supplemental oral PrEP to prevent infection and the selection of drug-resistant HIV (Landovitz, Li et al. 2020). Designing removable ultra-long-acting formulations, as disclosed herein, will address this critical limitation to significantly advance the technology.

Additionally, protection of women from unplanned pregnancy and infection by STIs remains imperative, and increased attention to MPTs is essential. Recent clinical trials have shown that the protective efficacy of antiretrovirals (ARVs) for prevention of HIV transmission correlates with adherence. Long-acting (LA) pre-exposure prophylaxis (PrEP) formulations that provide sustained drug release over weeks or months can potentially enhance compliance to prophylactic therapies and reduce the incidence of new HIV infections and unplanned pregnancy. Amongst LA formulations recently approved for HIV prevention, LA injectable formulation of cabotegravir (CAB) and rilpivirine (RPV) (Cabenuva®) has shown high acceptability among users compared to other methods and promising results in human Phase 3 studies supporting its recent approval for HIV treatment (Markowitz, Frank et al. 2017, Clement, Kofron et al. 2020). Moreover, injectable contraceptive use is highly acceptable in Africa, where HIV prevalence is highest, and has increased substantially over the past few decades. Despite their high acceptability among users, there are no injectable MPT formulations currently in development. This is mainly due to limitations of current injectable formulations utilizing nanoparticle suspensions, like CAB LA and DepoProvera®, which allow for injection of dense drug loads providing delivery of adequate doses in small volumes to produce sustained plasma concentrations. However, there are several concerns regarding the manufacturing and safety of these formulations. For example, because of how they are produced, two drugs cannot be combined into one formulation and require separate injections. Of importance, once administered, nanoparticle formulated LA injectable drugs cannot be removed. Therefore, in the event of breakthrough infection, toxicity, an allergic response, or pregnancy, the offending agent cannot be removed. For Cabenuva®, the inability to remove this injectable formulation requires a 4-week ‘lead-in’ regimen using oral cabotegravir and/or rilpivirine to fulfil current safety considerations. This technology also does not offer the ability to co-formulate two drugs in a single injection, which is why Cabenuva is administered as two separate injections of CAB and RPV. Similarly, when considering discontinuation of non-removable LA injectables, daily oral tenofovir disoproxil fumarate/emtricitabine (TDF/FTC) is necessary to ‘cover’ the entire ‘wash out’ period and to prevent seroconversion. This is an important consideration since ideally, LA injectable PrEP is aimed at mitigating lack of adherence to daily oral PrEP.

Many LA nano-based formulations are currently in development as LA HIV PrEP/ART including oral EFV/LPV (NANO-EFV/LPV, Phase I). However, there are still some limitations associated with these formulations, including high dose volume, long drug tail after treatment termination, complex manufacturing process, inability to co-formulate two or more drugs in a single injection, and inability to remove the injected dose once administered in the event of allergic or adverse reactions or pregnancy. Therefore, alternative injectable formulations that can deliver more than one drug in a single injection and can be removed to terminate the treatment hold great promise in eliminating these limitations.

In situ forming implants (ISFIs) may provide desirable properties for an ultra-long-acting CAB (or other drugs) formulation including long dosing intervals, small injection volumes, and retrievability. ISFIs comprise, in some embodiments, a hydrophobic and biodegradable polymer (e.g., poly(lactic-co-glycolic acid) (PLGA)), biocompatible water-miscible organic solvents (e.g., N-methyl-2-pyrrolidone (NMP) or dimethyl sulfoxide (DMSO)), and active pharmaceutical ingredients (APIs) that are co-formulated to generate a homogenous and syringeable liquid solution or suspension. Upon injection into the intramuscular or subcutaneous space, the water-miscible organic solvent diffuses into the aqueous environment, resulting in a phase inversion generating a solid or semi-solid depot comprising the API entrapped within the precipitated polymer matrix (Eliaz and Kost 2000, Agarwal and Rupenthal 2013, Parent, Nouvel et al. 2013, Thakur, McMillan et al. 2014). APIs are released from the depot via diffusion through the polymer matrix and via polymer bulk degradation over time.

As disclosed herein, a drug suspension in a polymer based biodegradable, removable in-situ forming implant has been developed. In-situ forming implants (ISFIs) by phase inversion techniques are defined as liquid polymer formulations that precipitate in-situ into a solid matrix through a process of phase inversion after injection into a subcutaneous or intramuscular environment. Poly-lactic-co-glycolic acid (PLGA) is a common FDA-approved polymer for ISFI systems in medical applications owing to its biodegradability, drug biocompatibility, suitable biodegradation kinetics and ease of processing. These ISFIs are comprised of only three components: biodegradable polymer (e.g. PLGA, PLA, PCL or other), a water-miscible biocompatible solvent (e.g. NMP, DMSO), and a drug(s). However, there are still some limitations using ISFI systems which include: 1) some excessive burst release (greater than about 20%) during phase inversion, 2) limited volume that can be administered due to the inherent toxicity of the organic solvent used; and 3) limited amount of drug that can be loaded within the allowed volume of injection into humans.

Disclosed herein for the first time is the development of a stable polymer based injectable drug suspension that can offer high drug loading, minimal burst release (e.g. less than about 5%), and ultra-LA release of antiretrovirals (or other any other drugs or pharmaceutical agents) alone or in combination (with another ARV or contraceptive). This process leads to superior control over initial burst release and drug release kinetics and duration. An important criterion to the formation of a stable suspension is the ability to push the drug concentration beyond its saturation concentration in the placebo ISFI formulation (i.e. polymer/solvent solution) without creating a phase separation between the polymer and solvent. In some cases, if the drug has low affinity for the polymer such as in the case of hydrophilic molecules, formulating the drug beyond its saturation solubility in an ISFI solution leads to a phase separation of the original placebo formulation and inability to form a stable suspension. Stability, microstructure, injectability, and release kinetics were defined in vitro and in vivo. This formulation is shown to be safe in mice and non-human primates and can release CAB for 6-11 months at levels that are above established benchmarks for PrEP protection in macaques and humans (664 ng/mL or 4×PA-IC₉₀). As disclosed herein, the extended release of CAB from the ISFI is associated with long-lasting protection against SHIV infection in a macaque model of PrEP that predicted clinical efficacy of CAB LA and other approved oral PrEP regimens. This study identifies a novel platform for the extended release of CAB at levels that are known to be associated with PrEP protection in humans.

The ISFI compositions disclosed herein can include one or more drugs as disclosed herein. The term “drug” can in some embodiments refer to a biologic, an active agent and/or a therapeutic compound, among other recognized terms of art, including but not limited to an antiviral, antibacterial, antifungal, contraceptive, prophylactic, anti-inflammatory, anticancer, analgesic, hormone, and combinations thereof. Moreover, by way of example and not limitation, a drug can comprise one or more drugs, optionally wherein the drug comprises biologics, active agents and/or therapeutic compounds, optionally wherein the drug is an antiretroviral drug, e.g. Cabotegravir (CAB), Dolutegravir (DTG), Doravirine (DOR), lamuvidine (3TC), and Islatravir (EFdA), emtricitabine (FTC), tenofovir disoproxil fumarate (TDF) or tenofovir alafenamide (TAF), optionally wherein the drug is a chemotherapeutic drug or agent, e.g. paclitaxel (PTX) and gemcitabine (Gem), optionally wherein the drug is a steroid, e.g. dexamethasone, cortisone, optionally wherein the drug is an opioids, e.g. oxycodone, hydrocodone, optionally wherein the drug is a contrast agent, e.g. radiopaque agents, fluorophores, radioactive agents, bioluminescent agents.

In some embodiments, the drug, active agent, etc. can be included in the ISFI at concentrations translatable to a human dose required to achieve a desired therapeutic effect. In some embodiments, such a concentration can be about 5 wt % to about 85 wt %, optionally about 10 wt % to about 75 wt %, about 15 wt % to about 70 wt %, about 20 wt % to about 65 wt %, about 25 wt % to about 60 wt %, about 30 wt % to about 55 wt %, optionally about 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, or 85 wt %. A “desired therapeutic effect” or the like can in some embodiments be used similar to “treating,” “treatment”, and “to treat” as defined herein, and can be used to indicate the production of beneficial or desired results, such as to alleviate symptoms, or eliminate the causation of a disease or disorder either on a temporary or a permanent basis, slow the appearance of symptoms and/or progression of the disorder, or prevent progression of disease. A desired therapeutic effect can refer to both therapeutic treatment and prophylactic or preventative measures, wherein the object is to prevent or slow down the development or spread of disease or symptoms. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total). “Treatment” or “desired therapeutic effect” can also refer to prolonging survival as compared to expected survival if not receiving treatment.

Additionally, in some embodiments the ISFI compositions disclosed herein can include one or more contrast agents, including but not limited to a contrast agent that is a radiopaque, e.g. barium sulphate. In some embodiments, such contrast agents can comprise a radioactive, e.g. iodide, gadolinium. In some embodiments, such contrast agents can comprise a fluorophore, e.g. fluorescent, indocyanine green, green fluorescent protein (GFP), m-cherry In some embodiments, such contrast agents can comprise a bioluminescent agent, e.g. luciferin.

To date, there are no reports on the use of a polymer-based drug suspension for ultra-long-acting delivery one or more drugs in a single injection.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Preparation of In-Situ PLGA-Based Drug Suspensions

The polymer based drug suspensions were prepared by: (1) Placebo formulation: 50:50 Poly(DL-lactide-co-glycolide) (PLGA), MW 10 or 27 kDa, or Poly(lactic acid) (PLA) MW 10 kDa, was mixed with N-methyl-2-pyrrolidone (NMP) or a combination of NMP and DMSO (e.g. 1:1 v/v) at various weight ratios of PLGA/solvent (w/w) and allowed to dissolve by continuous mixing at room temperature (Placebo). To assess the effect of PLGA/solvent ratio on a) drug loading capacity, b) drug suspension formation, c) implant macro-/microstructures, and d) drug release kinetics, placebo formulations containing 1:1, 1:1.5, 1:2, 1:3, and 1:4 w/w ratios of PLGA/solvent were prepared. (2) Drug loading: Drug (or combination drugs) (Cabotegravir (CAB), Dolutegravir (DTG), lamuvidine (3TC), Islatravir (EFdA), Doravirine (DOR), medroxyprogesterone acetate (MPA), entonogestrel (ENG)) was subsequently added to the PLGA/solvent placebo solution above its saturation concentration in the formulation and was allowed to stir at 37° C. overnight to form a stable suspension. (3) Depot formation: A sample from the resulting drug-loaded suspension formulation (30 μL, 30±3 mg) was injected into 200 mL of 0.01 M PBS pH 7.4 using a pipette and incubated at 37° C. for 24 h to produce a spherical implant, as an example. To assess the effect of co-solvent in the formulations on drug loading capacity, and release kinetics, the solvent containing 1:1 w/w ratio of NMP/dimethyl sulfoxide (DMSO) was prepared, then mixed with PLGA at 1:1, 1:1.5, 1:2, 1:3 or 1:4 w/w ratio of PLGA/solvent and formed PLGA implants following the aforementioned procedure.

### Example 2

### Drug Saturation Solubility Studies

The saturation concentration of each drug in various solvents and/or solvent combinations was determined. Drugs were then formulated in ISFI formulations containing 1:1, 1:1.5, 1:2, or 1:4 w/w ratios of PLGA/solvent, as exemplary starting points. For example, for DTG, 25 mg of DTG was added to individual vials containing 100 mg of solvent or PLGA/N-MP placebo ISFI solution. The mixture was mixed thoroughly using a vortex with short-warming for several cycles, and stirred at room temperature (RT), e.g. about 20-22° C., for about 24-48 h. Samples were subsequently centrifuged for 30 min at 13,000 rpm (Eppendorf Centrifuge 5417C, USA) to remove excess undissolved drug. Sample aliquots (1 mg, n=4) were collected from the saturated supernatant and diluted with acetonitrile (ACN). Drug concentration in the saturated aliquots was determined by HPLC analysis.

A reverse-phase HPLC analysis was carried out with a Finnigan Surveyor HPLC system (Thermo Finnigan, San Jose, California, USA) with a Photodiode Array (PDA) Plus Detector, auto-sampler, and LC Pump Plus. The stationary phase utilized for the analysis was a Inertsil ODS-3 column (4 μm, 4.6 Å˜150 mm, [GL Sciences, Torrance, CA]) maintained at 40° C. Chromatographic separation was achieved by gradient elution using a mobile phase consisting of 0.1% trifluoroacetic acid in water and ACN (H₂O/ACN 95:5 v/v). The flow rate was 1.0 mL/min and the total run time was 25 min for each 25 μL injection.

Drug saturation solubility test in various solvents/solvent combinations.

TABLE 1
Saturation solubility of antiretroviral drugs in various solvents (n = 4).

| | CAB Saturation |
|---|---|
| Solvent | Solubility (mg/mL) |
| NMP | 131.70 ± 3.66 |
| NMP:Gelucire 44/14 (9:1 w/w) | 134.21 ± 3.81 |
| NMP:DMSO (9:1 w/w) | 144.49 ± 1.99 |
| NMP:DMSO (1:1 w/w) | 167.12 ± 12.04 |
| NMP:DMSO (2:8 w/w) | 52.25 ± 6.39 |
| DMSO | 38.00 |
| (NMP:DMSO 1:1 w/w):Tween 20 (9:1 w/w) | 106.61 ± 3.05 |
| NMP:b-CD (6:4 w/w) | 81.75 ± 2.35 |
| NMP:HP-b-CD (6:4 w/w) | 89.58 ± 5.49 |
| NMP:HP-b-CD (9:1 w/w) | 158.22 ± 1.12 |
| PBS | 0.014 ± 0.001 |
| PBS + 2% Solutol | 0.06 ± 0.002 |

### Example 3

### Preparation and Optimization of Drug Suspension Formulations

Drug (for example but not limited to CAB, DTG, 3TC, EFdA, DOR, MPA, ENG) was loaded into the PLGA/solvent placebo solutions above its saturation concentration (Cₛₐₜᵤᵣₐₜᵢₒₙ) until a stable and syringeable suspension formulation was obtained. The three exemplary hydrophobic drugs (DTG, CAB, DOR) tested formed stable syringeable formulations, which resulted in formation of stable depots when injected into the aqueous release medium (PBS, pH 7.4) at 37° C. Of importance, but without being limited to any particular theory or mechanism of action, is the ratio of the polymer:solvent:drug that achieves high drug loading in a stable polymeric suspension formulation and which is correlated to the drug's saturation solubility in the solvent system. For example, the saturation solubility of DOR in NMP:DMSO (1:1 w/w) is, in some embodiments, 1.6-fold higher than that of CAB (Table 2). As such, to obtain a stable suspension formulation with DOR, the ratio of drug:polymer and drug:solvent needed to be about 1.7-fold lower than those for CAB (Table 3) in order to obtain a stable suspension (DOR 290 mg/mL; CAB 500 mg/L). On the other hand, the two hydrophilic drugs tested (3TC and EFdA) did not form successful suspensions when formulated above their saturation concentration in placebo ISFI formulations. These results also demonstrated that EFdA can be formulated as a suspension, however when injected into PBS at 37° C., the formulation did not form a stable spherical depot (Tables 4-6).

TABLE 2
Drug saturation solubility in solvents and solvent combinations.

| | Cabotegravir | | | |
|---|---|---|---|---|
| | sodium | Doravirine | Cabotegravir | Dolutegravir |
| | (CAB—Na salt) | (DOR) | (CAB) | (DTG) |
| | Saturation | Saturation | Saturation | Saturation |
| | Solubility | Solubility | Solubility | Solubility |
| Solvent | (mg/mL) | (mg/mL) | (mg/mL) | (mg/mL) |
| NMP | 47.5 ± 1.72 | 257.6 ± 3.8 | 131.7 ± 3.66 | 268.74 ± 7.25 |
| DMSO | 31.6 ± 0.96 | 204.6 ± 1.9 | 38.0 | 55 ± 4.5 |
| 1:1 NMP: | 49.1 ± 1.2 | 267.6 ± 5.1 | 167.12 ± 12.04 | 229.3 ± 1.6 |
| DMSO | | | | |
| PBS + 2% | 1.83 ± 0.7 | 0.08 ± 0.01 | 0.06 ± 0.002 | 0.56 ± 0.004 |
| Solutol | | | | |

TABLE 3
Correlation between drug (DOR, CAB) saturation solubility and ratio of drug/polymer
and drug/solvent required for a stable suspension formulation.

| | | | POLYMER: | Polymer:Solvent: | Polymer:Solvent: |
|---|---|---|---|---|---|
| DRUG | POLYMER | SOLVENT | SOLVENT | Drug (mg/mg/mg) | Drug (w/w/w) |
| CAB | 10 kDa 50:50 | 1:1 | 1:4 | 118:470:412 | 1:4:3.5 |
| | PLGA | NMP:DMSO | | | |
| | (Lactel) | | | | |
| DOR | 10 kDa 50:50 | 1:1 | 1:4 | 142:568:290 | 1:4:2 |
| | PLGA | NMP:DMSO | | | |
| | (Lactel) | | | | |

### Example 4

### Suspension Stability Studies

Eight (8) formulations were prepared and evaluated for stability. The formulations were placed in a water bath at 37° C. with shaking overnight and then vortexed for about 2 min upon removal. Sample aliquots (about 1 mg, n=4) were collected from each formulation and analyzed by HPLC to determine drug homogeneity distribution within the formulation. All suspension formulations were reevaluated for homogeneity after at least 24 h storage at room temperature (RT) to determine stability of suspensions at RT.

TABLE 4
CAB formulations investigated for suspension stability study.

| | | Homogeneity (mg/g) | | Ability to | Formed a |
|---|---|---|---|---|---|
| Formulation | Appearance | 0 h | >24 h | Resuspend | Depot? |
| 100 mg/g CAB 1:1.5 | Translucent | 70.7 ± 4.39 | 42.6 ± 14.7 | Difficult | Yes |
| PLGA:(NMP/DMSO, 1,1) | Suspension | | | | |
| 200 mg/g CAB 1:1.5 | Opaque | 135.5 ± 8.43 | 131.2 ± 17.2 | Difficult | Yes |
| PLGA:(NMP/DMSO, 1,1) | Suspension | | | | |
| 450 mg/g CAB 1:6 | White | 372.1 ± 26.9 | 397.2 ± 45.3 | Very Easy | Yes |
| PLGA:(NMP/DMSO, 1,1) | Suspension | | | | |

TABLE 5
DTG formulations for suspension stability study.

| | | Homogeneity | | Ability to | Formed a |
|---|---|---|---|---|---|
| Formulation | Appearance | 0 h | >24 h | Resuspend | Depot? |
| 100 mg/g DTG | Solution | 101.7 ± 2.15 | N/A | N/A | Yes |
| 1:1.5 PLGA:NMP | | | | | |
| 200 mg/g DTG | Solution with | 207.8 ± 5.42 | 136.3 ± 14.1 | Very | Yes |
| 1:1.5 PLGA:NMP | Particles | | | Difficult | |
| 250 mg/g DTG 1:4 | Translucent | 232.0 ± 8.86 | 115.3 ± 6.53 | Very | Yes |
| PLGA:NMP | Suspension | | | Difficult | |
| 300 mg/g DTG 1:4 | Opaque | 228.4 ± 13.5 | 263.4 ± 19.4 | Easy | Yes |
| PLGA:NMP | Suspension | | | | |

TABLE 6
Hydrophilic drugs (3TC & EFdA) formulations for suspension stability study. Both 3TC and
EFdA homogeneity sample showed two peaks on the HPLC. These double peaks were not seen in
the standards, indicating that the drug may be interacting with the polymer or solvent.

| | | | | | | | 24 h- |
|---|---|---|---|---|---|---|---|
| | | Polymer:Solvent | | Concentration | | Depot | Burst |
| Polymer | Solvent | Ratio | Drug | (mg/g) | Observation | Forming | (%) |
| 27 kDa 50:50 | 1:1 w/w | 1:2 | 3TC | 250 | Thick | NO | N/A |
| PLGA | NMP:DMSO | | | | paste | | |
| | | | | | consistency | | |
| 27 kDa 50:50 | 1:1 w/w | 1:2 | 3TC | 200 | Paste | NO | N/A |
| PLGA | NMP:DMSO | | | | consistency | | |
| 27 kDa 50:50 | 1:1 w/w | 1:2 | 3TC | 160 | Tick | NO | N/A |
| PLGA | NMP:DMSO | | | | translucent | | |
| | | | | | suspension | | |
| 6:1 w/w | NMP | 1:2 | EFdA | 167 | Stable | YES | 64 |
| PLA (10 | | | | | suspension | | |
| kDa):Pluronic | | | | | | | |
| L61 | | | | | | | |
| 9:1 w/w | NMP | 1:2 | EFdA | 200 | Stable | YES | 58 |
| PLA (10 | | | | | suspension | | |
| kDa):Pluronic | | | | | | | |
| L61 | | | | | | | |

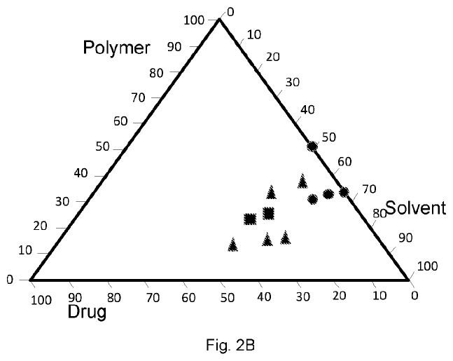

Ternary phase diagrams. Ternary phase diagrams were generated for CAB and DTG ISFI suspensions to determine the areas of stable suspensions that can result in formation of stable solid depots upon injection into an aqueous medium. Phase diagrams were generated based on existing data and proposed scenarios based on theoretical assumptions (FIG. 2).

TABLE 7
CAB ISFI formulations used to generate a ternary phase diagram

| | | Content | | | | | |
|---|---|---|---|---|---|---|---|
| | | Drug | | | | | |
| | | Conc. | | | Content (wt %) | | |
| Formulation | Appearance | (mg/g) | Polymer | Solvent | Polymer | Solvent | Drug |
| 100 mg/g CAB 1:2 PLGA:(NMP/DMSO, 1/1) | Solution | 100 | 1 | 2 | 30.00 | 60.00 | 10.00 |
| 200 mg/g CAB 1:2 PLGA:(NMP/DMSO, 1/1) | Suspension | 200 | 1 | 2 | 26.67 | 53.33 | 20.00 |
| 250 mg/g 1:2 PLGA:(NMP/DMSO, 1/1) | Suspension | 250 | 1 | 2 | 25.00 | 50.00 | 25.00 |
| 400 mg/g CAB 1:4 PLGA:(NMP/DMSO, 1/1) | Suspension | 400 | 1 | 4 | 12.00 | 48.00 | 40.00 |
| 300 mg/g CAB 1:4 PLGA:(NMP/DMSO, 1/1) | Suspension | 300 | 1 | 4 | 14.00 | 56.00 | 30.00 |
| 100 mg/g CAB 1:1.5 PLGA:(NMP/DMSO, 1, 1) | Translucent | 100 | 1 | 1.5 | 36.00 | 54.00 | 10.00 |
| | Suspension | | | | | | |
| 200 mg/g CAB 1:1.5 PLGA:(NMP/DMSO, 1, 1) | Suspension | 200 | 1 | 1.5 | 32.00 | 48.00 | 20.00 |
| 450 mg/g CAB 1:6 PLGA:(NMP/DMSO, 1, 1) | Suspension | 450 | 1 | 6 | 7.86 | 47.14 | 45.00 |

TABLE 8
DTG ISFI formulations used to generate a ternary phase diagram

| | | Content | | | | | |
|---|---|---|---|---|---|---|---|
| | | Drug Conc. | | | Content (wt %) | | |
| Formulation | Appearance | (mg/g) | Polymer | Solvent | Polymer | Solvent | Drug |
| 50 mg/g DTG 1:2 PLGA:NMP | Solution | 50 | 1 | 2 | 31.67 | 63.33 | 5.00 |
| 100 mg/g DTG 1:2 PLGA:NMP | Solution | 100 | 1 | 2 | 30.00 | 60.00 | 10.00 |
| 250 mg/g DTG 1:2 PLGA:NMP | Suspension | 250 | 1 | 2 | 25.00 | 50.00 | 25.00 |
| 300 mg/g DTG 1:2 PLGA:NMP | Suspension | 300 | 1 | 2 | 23.33 | 46.67 | 30.00 |
| 250 mg/g DTG 1:4 PLGA:NMP | Suspension | 250 | 1 | 4 | 15.00 | 60.00 | 25.00 |
| 300 mg/g DTG 1:4 PLGA:NMP | Suspension | 300 | 1 | 4 | 14.00 | 56.00 | 30.00 |
| 400 mg/g DTG 1:4 PLGA:NMP | Suspension | 400 | 1 | 4 | 12.00 | 48.00 | 40.00 |
| 100 mg/g DTG 1:1.5 PLGA:NMP | Solution | 100 | 1 | 1.5 | 36.00 | 54.00 | 10.00 |
| 200 mg/g DTG 1:1.5 PLGA:NMP | Suspension | 200 | 1 | 1.5 | 32.00 | 48.00 | 20.00 |

### Example 5

### Scanning Electron Microscopy (SEM) Imaging and Analysis

Microstructures of solid implants were evaluated by scanning electron microscopy (SEM). To investigate the effect of PLGA/NMP weight ratio on drug distributions and the microstructure of the depots prepared with CAB or DTG only and CAB or DTG in combination with a contraceptive drug (etonogestrel or depot medroxyprogesterone (DMPA). Depots were prepared by injecting formulation suspensions (25 μL) containing DTG (100 mg/mL) or CAB (400 mg/mL) in 1:4 PLGA/(NMP:DMSO 1:1) (PLGA MW 10 kDa) into 200 mL of PBS and incubating for 72 h at 37° C. The resulting solid depots were removed from the PBS, flash frozen with liquid nitrogen, and then lyophilized for 24 h (SP VirTis Advantage XL-70, Warminster, PA). The lyophilized samples were subsequently fractured and mounted on an aluminum stub using carbon tape, and sputter coated with 5 nm of gold-palladium alloy (60:40) (Hummer X Sputter Coater, Anatech USA, Union City, CA). The coated samples were imaged using a Zeiss Supra 25 field emission scanning electron microscope with an acceleration voltage of 5 kV, 30 m aperture, and average working distance of 15 mm (Carl Zeiss Microscopy, LLC, Thornwood, NY).

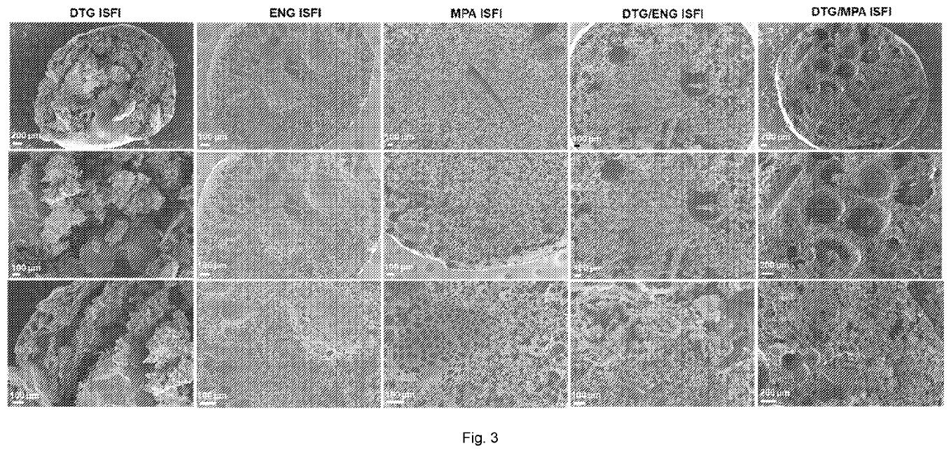

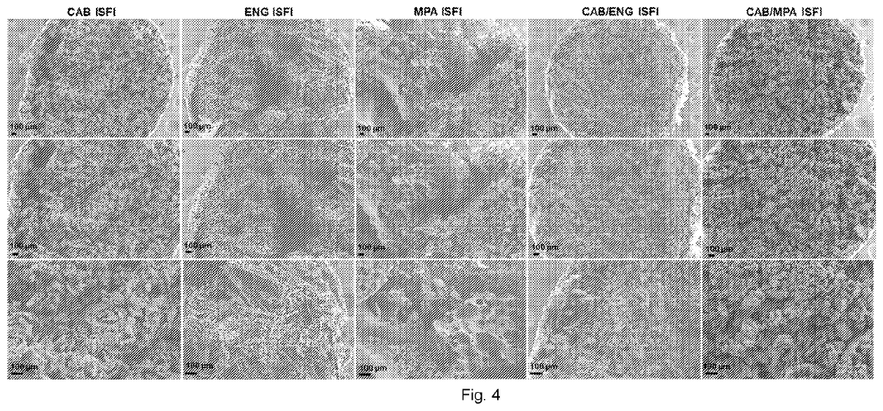

From the SEM images, one can clearly distinguish the drug crystals from the polymeric matrix (FIG. 3, 4). In addition, due to the higher drug concentration in the CAB ISFIs (FIG. 4), the microstructure of the CAB containing implants show a higher density of crystals (drug) compared to the DTG ISFIs (FIG. 3).

### Example 6

### In Vitro Drug Release Studies

Drug release kinetics from various implant formulations was evaluated by incubating solid implants (25 mg±5 mg) into 200 mL of release medium under sink conditions (0.01 M PBS pH 7.4 with 2% solutol HS) at 37° C. for up to 6 months. Sample aliquots (1 mL) were collected at various time points and replaced with fresh release medium. The release medium was completely removed and replaced with fresh medium every week to maintain sink conditions. Drug concentration in the release samples was quantified by HPLC using the method described above. Cumulative drug release was calculated from the HPLC analysis and normalized to the total mass of drug in the implant. All experiments were performed in quadruplicate.

### Example 7

### Effect of Drug Loading on In Vitro Release Kinetics

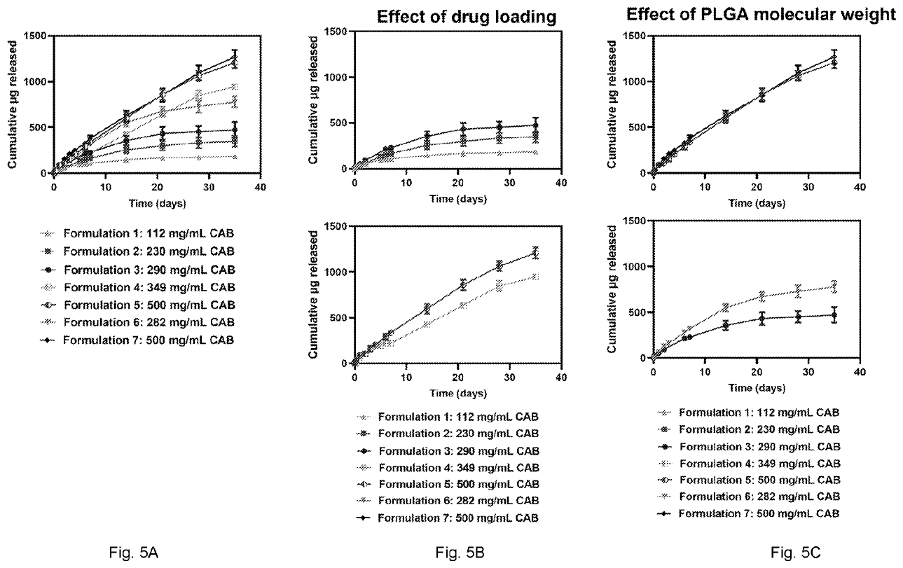

ISFIs were prepared with 1:2 w/w PLGA:(NMP/DMPS 1:1) (PLGA MW 27 kDa, 50:50 LA/GA). CAB was loading at 100 mg/g, 200 mg/g or 250 mg/g in the placebo ISFI to investigate the effect of CAB loading on its release kinetics. Results show that the burst release in the first 24 h was significantly lower for the drug suspensions (200 and 250 mg/g CAB ISFIs) compared to the solution (100 mg/g ISFI) formulation (FIG. 5B). Results also showed that the release rates were higher at higher drug loading with 5.31 μg/d, 2.8 μg/d, and 1.45 μg/d for CAB 250 mg/g, 200 mg/g and 100 mg/g ISFIs, respectively.

### Example 8

### Effect of Polymer MW on Drug Release Kinetics

To investigate the effect of polymer MW on CAB release kinetics, two CAB suspension ISFIs were prepared using PLGA (50:50 LA/GA) with MW of 10 kDa and 27 kDa. CAB was loaded at a constant concentration of 250 mg/g as a stable suspension formulation in a 1:2 PLGA/solvent (solvent=NMP/DMSO 1:1 w/w). Results showed that PLGA MW did not have an effect on the initial burst release of CAB within the first 24 h and both formulations had a very low burst of about 1% (FIG. 5C). Results also showed that CAB exhibited faster release rate when formulated in PLGA MW 10 kDa at 23. 55 μg/d compared to 13.53 μg/d for PLGA MW 27 kDa. Interestingly, when formulated at a higher concentration of 400 mg/g in the aforementioned formulations, the release rates were comparable and not statistically different (FIG. 5C).

### Example 9

### Effect of Formulation Composition on Drug Release Kinetics

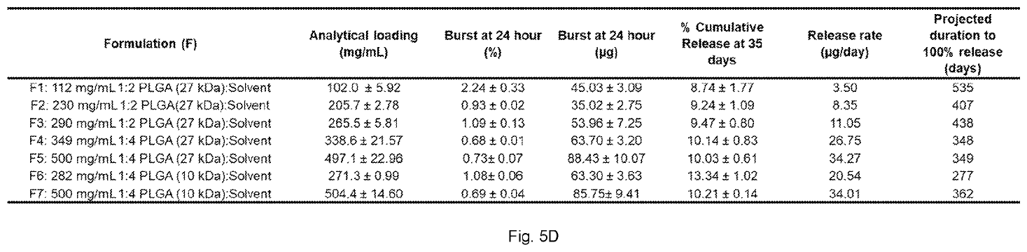

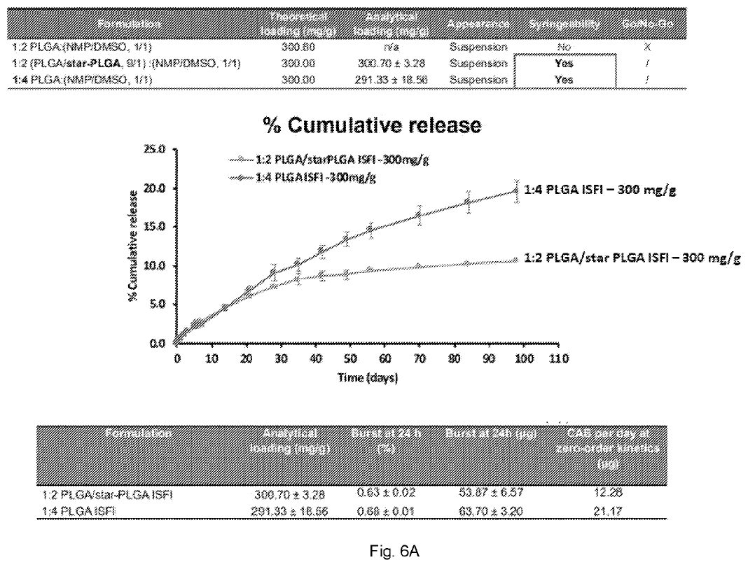

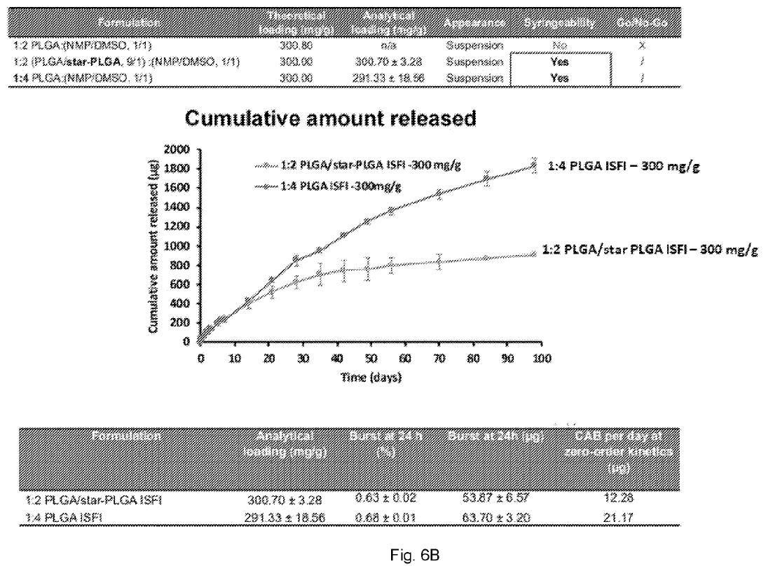

The effect of formulation composition and addition of rate controlling polymers like star-PLGA (5-arm 50:50 LA/GA PLGA MW 54 kDa) on drug release kinetics were also investigated. Three formulations were prepared using NMP/DMSO (1:1) as a co-solvent system, PLGA (50:50 LA/GA MW 27 kDa) and PLGA/star-PLGA (9:1 w/w) polymer combination. CAB was loaded at a constant concentration of 300 mg/g in all three formulations. Results showed that when formulated in a 1:2 PLGA/(NMP:DMSO) ISFI, CAB formed a stable suspension however the viscosity of this formulation was too high leading to a non-syringeable formulation (FIGS. 6A and 6B). Results also showed that addition of star-PLGA with linear PLGA at a 9:1 linerar/star-PLGA resulted in a syringeable suspension ISFI of CAB loaded at 300 mg/g. These results show that star-PLGA, in some embodiments, acts as a plasticizer by reducing the viscosity of the suspension ISFI when used in combination with linear PLGA. In addition, increasing the solvent ratio from 1:2 to 1:4 PLGA/(NMP:DMSO), in some embodiments, resulted in a syringeable suspension ISFI of CAB loaded at 300 mg/g. Using higher solvent ratios naturally reduces the viscosity of the formulation. With respect to drug release kinetics, results show that at higher solvent/polymer ratio (1:4 PLGA/(NMP:DMSO)), CAB exhibited higher release rates with 21.17 μg/d compared to lower release rates in the 1:2 PLGA/(NMP:DMSO) suspension ISFI at 12.28 μg/d (FIGS. 6A and 6B).

### Example 10

### In Vitro Release of DOR ISFIs

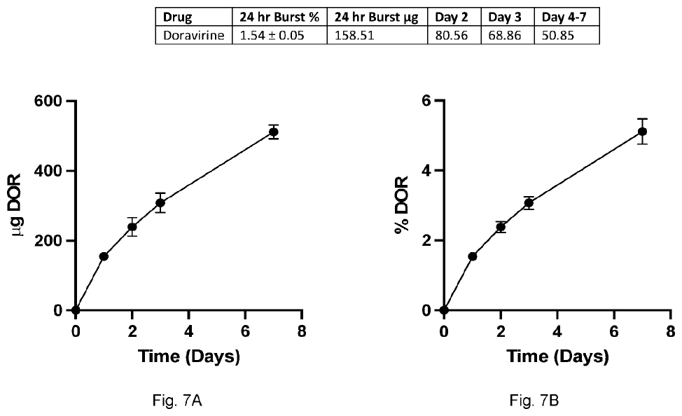

Doravirine (DOR, NNRTI) was successfully formulated as a stable suspension formulation (1:4 PLGA/(NMP:DMSO); 290 mg/g) and in vitro release studies showed that like CAB, DOR exhibited very low burst release (<2% in 24 h) and sustained drug release over the first week (3% at d3, last timepoint analyzed) (FIGS. 7A & B). These results, along with others herein, demonstrate that the disclosed ISFIs are suitable for any desired drug, pharmaceutical or active agent, particularly where sustained release is necessary.

### Example 11

### In Vivo Safety Studies in BALB/c Mice

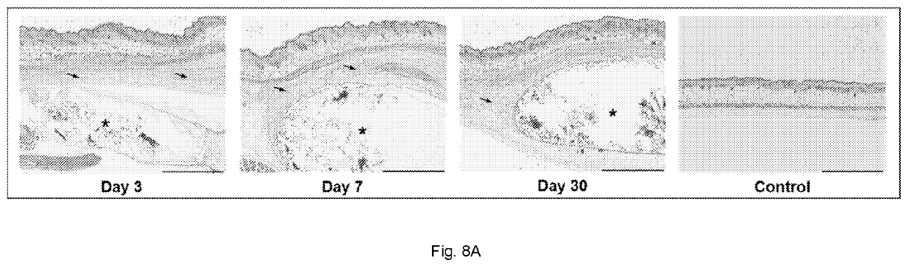

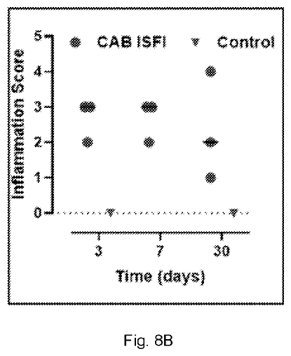

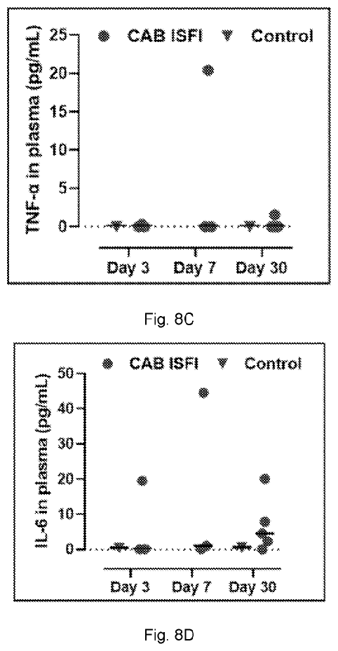

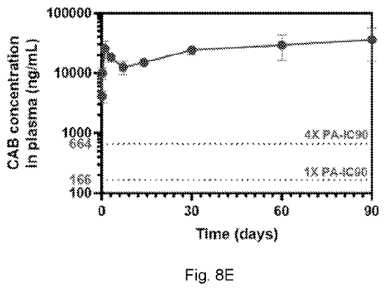

A 30-day in vivo safety study of CAB ISFI (CAB used as proof of concept but other drugs/APIs expected to work similarly) was conducted with female BALB/c mice to assess local and systemic inflammation post-injection. Results from the study showed that the CAB ISFI was well-tolerated, and mice did not show any signs of overt toxicity, behavioral changes, or weight loss. Histopathological analysis of excised implant and surrounding subcutaneous tissue demonstrated that the CAB ISFI exhibited mild to moderate local inflammation shown by infiltrated immune cells around the depot (FIG. 8A). At day 3 and 7, the median skin microscopic inflammation score was 3 (moderate inflammation) likely due to the initial immune response to the injection and decreased by day 30 in 2 out of the 3 mice tested (FIG. 8B).

Systemic inflammation was assessed by enzyme-linked immunosorbent assay (ELISA) to quantify TNF-α and IL-6 proinflammatory cytokines in plasma. Results showed no systemic acute or chronic inflammation. TNF-α ranged between 0-20 pg/mL and was comparable to the no injection control group (p>0.05) (FIG. 8C). IL-6 levels in plasma ranged between 0-45 pg/mL (FIG. 8D) and levels were comparable to those seen in the no injection control group [p>0.05 (2-way ANOVA with multiple comparisons)]. Variability in inflammation scores or proinflammatory cytokines levels can be attributed to interindividual variability, or hormone-cycle variability in mice (LaMarca, Chandler et al. 2007). Overall, these results demonstrated that CAB ISFIs were generally well-tolerated and considered safe with no overt signs of toxicity or chronic inflammation. For at least these reasons ISFIs with other drugs are expected to be equally well-tolerated.

### Example 12

### In Vivo Pharmacokinetic Studies in BALB/c Mice

Pharmacokinetic (PK) studies were carried out in female BALB/c mice over 90 days to assess in vivo drug release kinetics of CAB ISFI. CAB plasma concentrations were quantified using a high-performance liquid chromatography-tandem mass spectrometry LC/MS-MS method and were plotted over 90 days (FIG. 8E). Furthermore, CAB release kinetics were assessed against three mathematical models (zero order, first order, and diffusion-controlled (Tiwari et al. 2010). It was determined that the observed CAB in vivo release in mice best fits a zero-order model (zero order model R²=0.97; first order model R²=0.87; diffusion controlled model R²=0.86) over the 90-day PK study duration. Moreover, CAB concentration in plasma was between 3.7-75-fold greater than the 4×PA-IC₉₀ (664 ng/mL (Trezza, Ford et al. 2015)) for all mice during the entire 90 day study (FIG. 8E).

### Example 13

### Assessment of CAB ISFI Injectability

Based on the promising safety and PK data in BALB/c mice, the CAB ISFI formulation was selected for evaluation of safety, PK and efficacy in rhesus macaques. However, since the injection volume for macaques (two 1 mL injections) would be much higher than for mice (50 μL), it was essential to ensure formulation injectability of large volumes in vitro prior to scaling to the macaque studies. To assess injectability of the optimized CAB ISFI formulation, we utilized polyacrylamide hydrogels (Hernandez, Gawlik et al. 2016). Polyacrylamide hydrogels have been shown to mimic the mechanical properties of in vivo subcutaneous tissue at the injection site, and to elicit better correlation to in vivo release for ISFIs rather than standard in vitro release methods by direct injection into a PBS bath (Hernandez, Gawlik et al. 2016, Manaspon, Hernandez et al. 2017). Assessing injectability was essential for the CAB ISFI formulation due to its fast phase inversion property upon injection, which is attributed to the organic solvents' high miscibility with water and low PLGA MW (Parent, Nouvel et al. 2013). If the phase inversion is too quick, the formulation could solidify between the syringe and needle and block the flow of injection.

As such, the injectability of several placebo formulations with varying polymer to solvent ratios and PLGA MW as well as the optimized CAB ISFI formulation (1:4 w/w PLGA (10 kDa):solvent) were investigated. Injectability of formulations into polyacrylamide hydrogels was investigated with 16 gauge (G), 18G, and 19G needles with 1 mL injection volume. 1 mL injection into the hydrogel matrix with a 19G needle of the 1:4 w/w PLGA (10 kDa):solvent placebo or CAB ISFI formulation was not successful due to rapid phase inversion and fast depot formation leading to obstruction of formulation flow through the needle. This was likely attributed to the combination of low MW PLGA (10 kDa) and high amounts of solvent (1:4 w/w PLGA:solvent). On the other hand, 1 mL of placebo ISFIs prepared with higher PLGA MW (27 kDa) or lower amounts of solvent (1:3 and 1:2 w/w PLGA:solvent) were successfully injected into the hydrogel matrix. In addition, 1 mL injection of the optimized CAB ISFI (500 mg/mL 1:4 PLGA (10 kDa):solvent) into the hydrogel matrix was successfully achieved with an 18G or 16G eedle. Based on these results, a 16G needle was used to easily administer CAB ISFI formulation in macaque studies.

### Example 14

### CAB Release from ISFIs in Rhesus Macaques

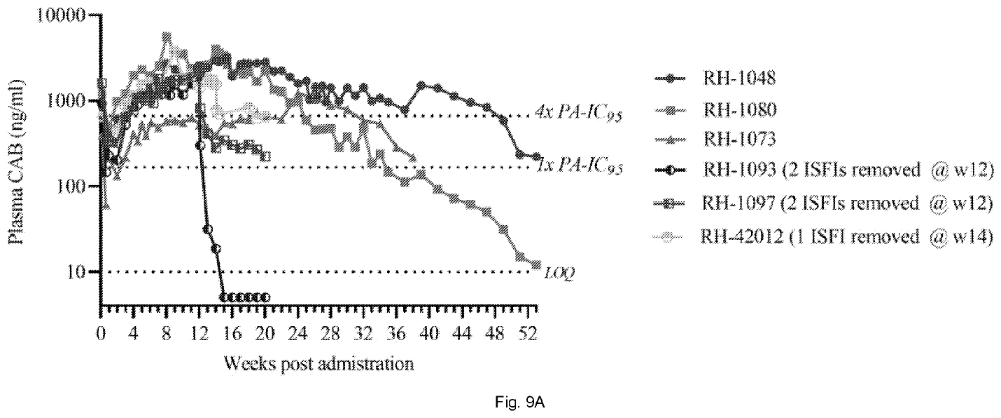

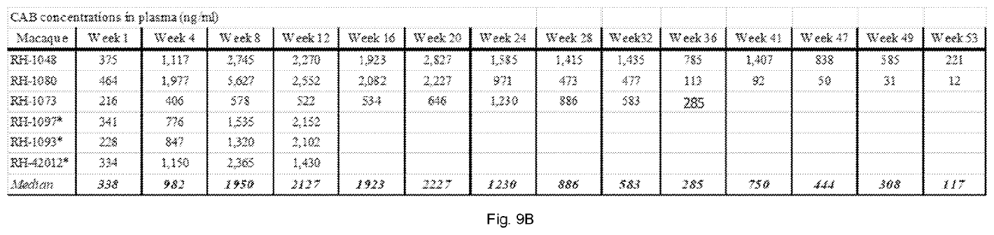

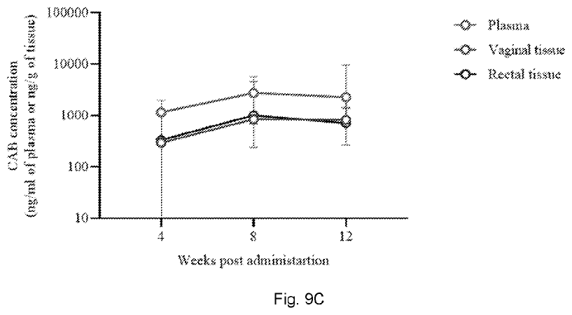

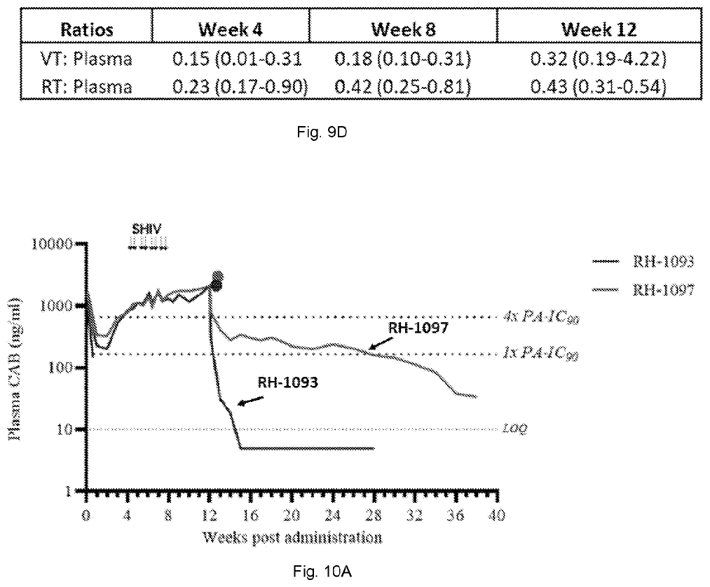

CAB ISFI was administered to 6 female rhesus macaques. All the animals received two separate 1 mL injections (total of 1000 mg of CAB) with the exception of one macaque (RH-1080) which received a 1.0 mL and 0.5 ml injection of ISFI or a total of 750 mg of CAB. On a per weight basis, animals received between 72.8 and 143.9 mg/kg of CAB (median=113.8 mg/kg). Macaque RH-42012 was a SHIV-infected and otherwise healthy animal from a separate study and was included for PK purposes only. FIG. 9A shows longitudinal concentrations of CAB in plasma. Overall, all the macaques achieved plasma CAB concentrations above the 4×PA-IC₉₀ by week 4 with the exception of RH-1073, which achieved benchmark concentrations at week 24 (1,230 ng/ml). Median (range) plasma concentrations of CAB at weeks 4, 8 and 12 were 982 [406-1,977], 1,950 [578-5,627] and 2,127 [522-2,552] ng/mL, respectively, or about 1.5- to 3.2-fold above the 4×PA-IC₉₀. Removal of the two CAB ISFIs in macaques RH-1097 and RH-1093 at week 12 resulted in a 7- and 48-fold reduction in plasma CAB levels at 72 hours and about 10-100-fold decline at two weeks post removal, respectively (FIG. 9A). Likewise, removal of one of the two ISFIs that remained palpable in macaque RH-42012 at week 14 resulted in ˜2-fold decline in plasma CAB within a week (1,550 to 765 ng/mL); CAB concentrations still remained above the 4×PA-IC₉₀ for an additional 3 weeks. In the remaining 3 animals with intact CAB ISFIs, the median plasma CAB concentrations at weeks 16, 20, 24, and 28 were 1,923 [534-2,082], 2,227 [646-2,827], 1,230 [971-1,585], and 886 [473-1,415] ng/mL, respectively, or 1.3- to 3.4-fold above the 4×PA-IC₉₀ (FIG. 9B). Notably, the plasma CAB levels in one animal (RH-1048) remained above the 4×PA-IC₉₀ at week 47 (838 ng/mL). Overall, these results demonstrate that two 1 mL injections of the optimized CAB ISFI formulation can release CAB in macaques at levels above the threshold for protection for up to 6 to 11 months.

CAB concentrations were also measured in vaginal and rectal tissues at weeks 4, 8 and 12. FIG. 9C shows that CAB was consistently detected in both tissues, with the only exception of macaque RH-1048 which had undetectable CAB in vaginal tissues at week 4. Median CAB concentrations in rectal tissues increased approximately 3-fold between week 4 and week 8 (333 to 1,004 ng/g, respectively) and slightly declined by week 12 (713 ng/g). Median CAB concentrations in vaginal tissues also increased about 2.8-fold from weeks 4 to 8 (293 to 849 ng/g, respectively) and remained at 823 ng/g at week 12. Tissue to plasma ratios remained stable overtime and were similar in the vaginal (median=0.18 (0.15-0.32) and the rectal compartment (median=0.42 (0.23-0.43)).

### Example 15

### Efficacy of CAB ISFI Against Rectal SHIV Infection

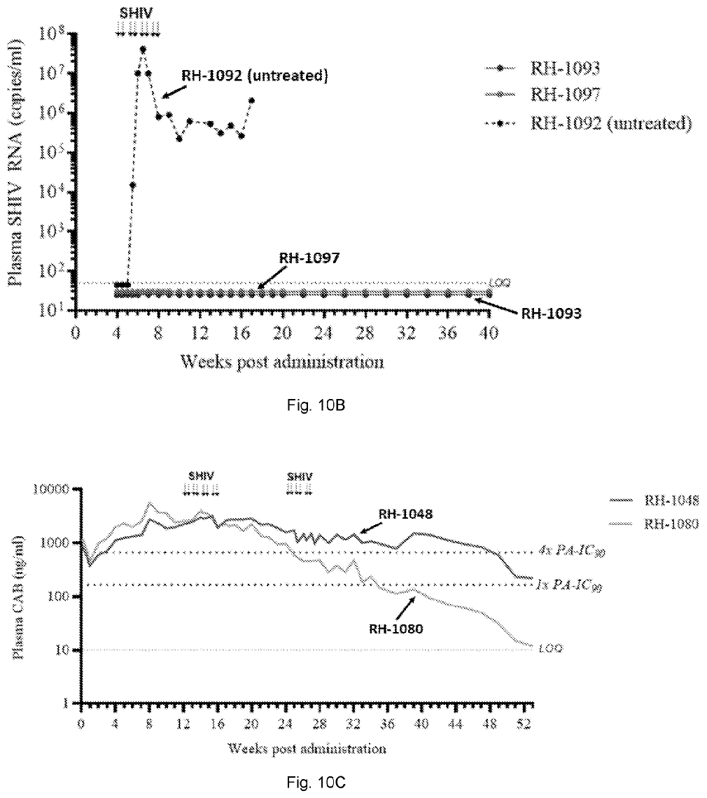

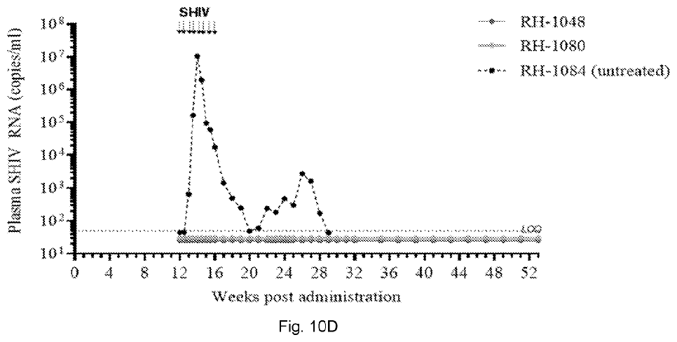

To investigate if CAB delivered from ISFIs could confer rectal protection, a series of SHIV challenge experiments were performed at different times after implantation. Short-term protection was first evaluated in two macaques (RH-1093 and RH-1097) challenged twice-weekly between weeks 4 and 8 (FIG. 10A). Both animals were protected against SHIV infection as opposed to an untreated real time control (RH-1092) that was challenged during the same period and infected after a single SHIV exposure. Long-term protection was evaluated in two additional macaques (RH-1048 and RH-1080) that were exposed twice per week to SHIV between weeks 12 and 16 for a total of 8 rectal challenges (FIG. 10B). One animal (RH-1048) further maintained plasma CAB levels above 4×PA-IC₉₀ and received additional 6 SHIV challenges between weeks 24 and 27 for a total of 14 rectal SHIV exposures. Both CAB treated animals were protected from infection while an untreated real time control (RH-1084) was infected after a single SHIV exposure (FIG. 10B). Overall, a single ISFI treatment provided protection during a cumulative of 38 SHIV exposures that was long-lasting for up to 27 weeks.

### Example 16

### Safety and Tolerability of CAB ISFI in Rhesus Macaques

To assess safety and tolerability, the area surrounding the implants were examined each week for up to 12 weeks or until the implant was removed. This assessment included the 6 macaques that received two injections for a cumulative analysis of 12 implantation sites and a total of 144 clinical observations. Based on the Draize scale, all implantation sites were unremarkable and showed no signs of local skin reactions during the 12-week study period (FIG. 11A). A semiquantitative histopathological assessment was also done on skin biopsies collected from three macaques during implant removal (12 to 14 weeks post implantation). Skins biopsies had no to minimal lymphoplasmacytic infiltrates, present among all animals, including the control, and is not associated with the implant, bacterial infection or residual implant material in any of the tissue sections (FIG. 11B).

### Example 17

### Residual Drug Quantification, Biodegradation, and Estimated Release Rates in BALB/c Mice and Rhesus Macaques

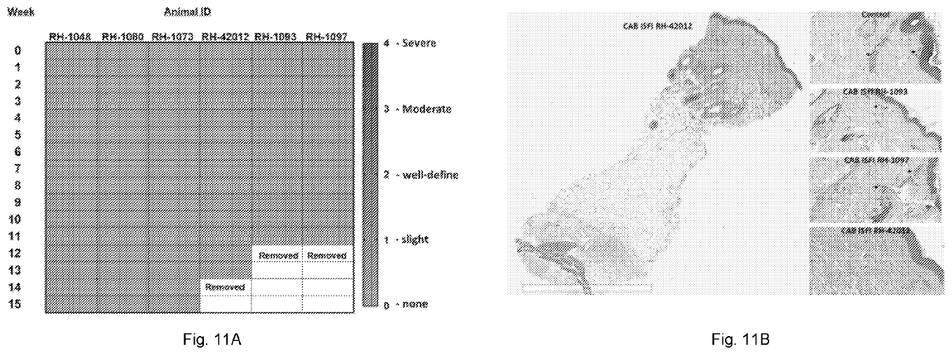

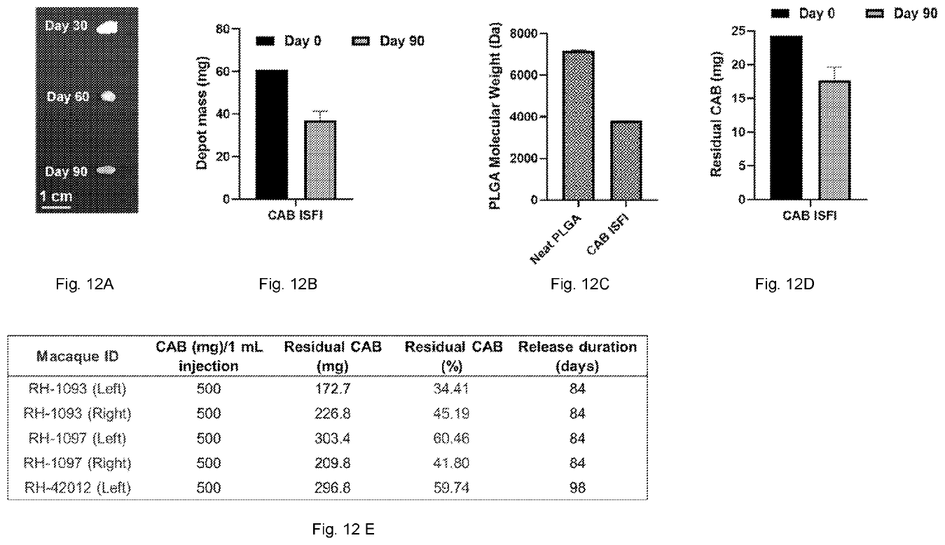

To assess retrievability of implants post in vivo studies, CAB ISFIs were removed from mice at days 30 (n=3), 60 (n=3), and 90 (n=5 (n=3 implants were used to quantify residual drug, n=2 implants were used to determine PLGA degradation) post administration by making a small skin incision at euthanasia. ISFIs were successfully removed from all animals with no fibrotic tissue surrounding the depot (FIG. 12A). Depots removed at day 90 were further processed to evaluate polymer degradation and residual CAB concentrations. Results from these analyses showed a 38.9±6.9% loss in depot mass (FIG. 12B) and 47.2±0.07% PLGA degradation after 90 days in vivo (FIG. 12C). Importantly, there was 72.6±8.2% of CAB remaining in the implants retrieved from mice at day 90 demonstrating that CAB release from the ISFI can be sustained beyond 90 days (FIG. 12D).

Furthermore, both CAB ISFIs were removed from 2 macaques (RH-1097 and RH-1093) at day 84 and one ISFI removed from a third macaque (RH-42012) at day 98 post-injection. As shown in FIG. 12E, there was an average of 48.32±11.44% CAB remaining per implant after depot removal.

### Example 18

### Translation of CAB ISFI NHP PK to Clinical Dosing in Humans

Individual CAB clearance rates were estimated based on the extravascular PK profile for our 6 macaques dosed with 1.5-2 mL subcutaneous CAB ISFI [median (IQR)=15.9 (9.1-25.2) mL/(hr*kg)] and for 9 historical reference macaques dosed with 50 mg/kg intramuscular CAB LA at 7 and 1 days before PK sampling [median (IQR)=12.4 (11.7-14.3) ml/(hr*kg)] (Spreen, Lowry et al. 2015). Input rates for these two long-acting formulations were derived by multiplying observed plasma concentration by respective animals' estimated clearance rate corrected for weight at time of administration in our 6 ISFI treated macaques or assumed weight of 8 kg in historical reference macaques (FIG. 13). Target input rates for effective clinical dosing were estimated using mean parameters from a published CAB population PK model (CL=151 mL/hr, V2=5270 mL, Q=507 mL/hr, and V3=2430 mL) (FDA 2021) to simulate human plasma concentrations at various IV infusion rates. Input rates of 3 and 0.75 mg/day achieved plasma concentrations above the 4×PA-IC₉₀ and 1×PA-IC₉₀, respectively. The median CAB ISFI input rates observed in our macaque study exceeded this 3 mg/day clinical efficacy threshold by day 28 out to day 140 post-administration. Assuming input rate increases proportionally with injection volume, a 3 mL CAB ISFI injection would achieve this threshold by day 21 out to day 168 or 5.6 months post administration. In comparison to the macaques dosed with two 50 mg/kg intramuscular CAB LA injections 6 days apart, CAB ISFIs maintained rates above the predicted protective threshold for a median of 97 extra days (FIG. 13B).

### Example 19

### CAB MPT ISFI In Vitro Release Studies

In vitro release studies with CAB MPT ISFIs (CAB/ENG and CAB/MPA) and single-drug ISFIs (CAB ISFI, MPA ISFI, and ENG ISFI) were conducted to (1) assess feasibility of co-formulating CAB with MPA or ENG in an ISFI (MPT), (2) determine any drug-drug interactions, and (3) to assess target in vitro release rates and target release duration (>90 days).

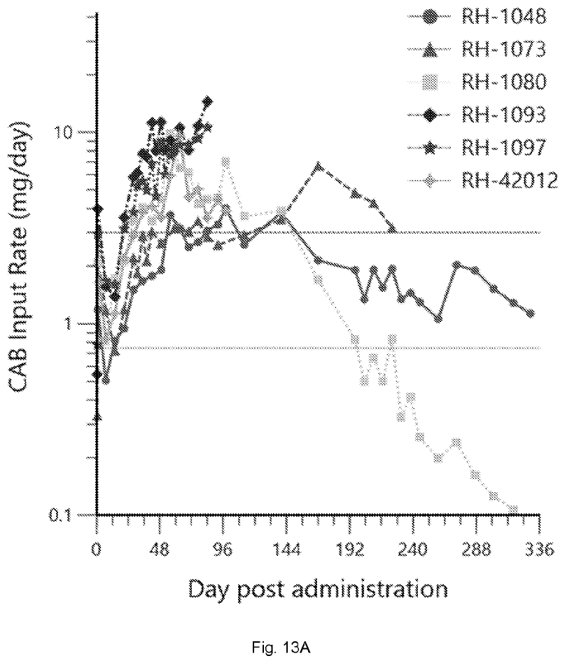

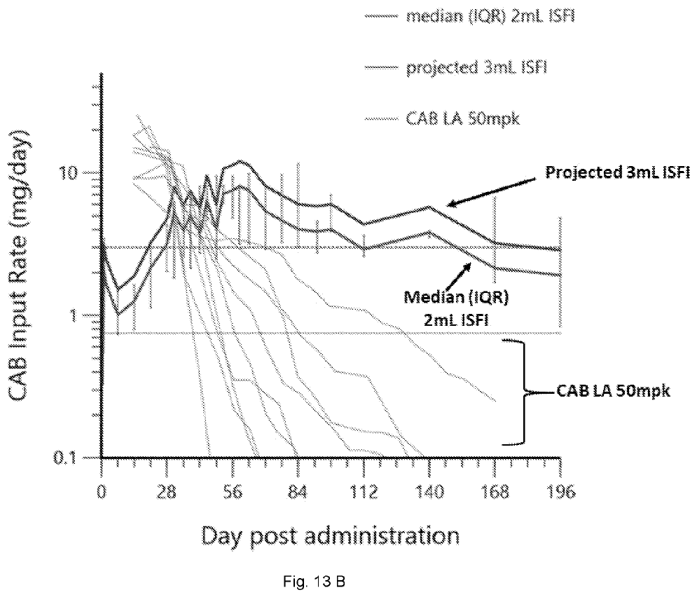

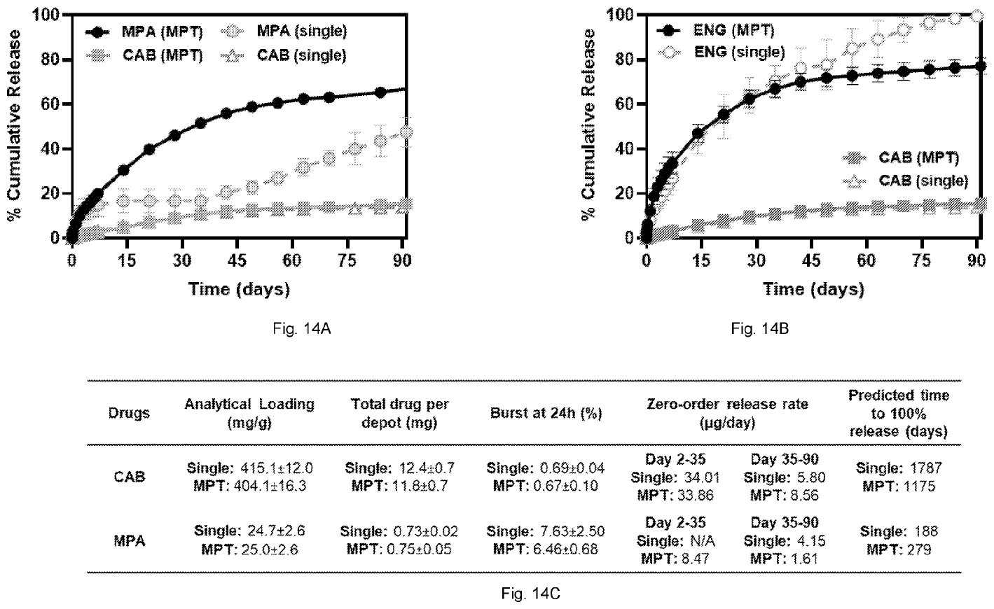

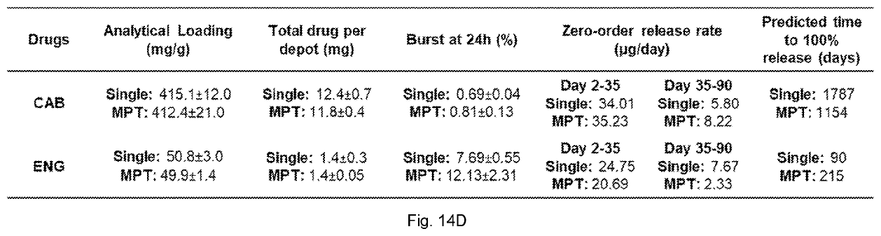

FIG. 14 demonstrates the cumulative in vitro release kinetics of CAB/MPA ISFIs (FIGS. 14A and 14C) and CAB/ENG ISFIs (FIGS. 14B and 14D). In vitro release of single-drug ISFIs vs. MPT ISFIs were evaluated to assess drug-drug interactions. All ISFI liquid suspensions were stable and homogenous, elicited <15% burst release, sustained release for 90 days, and above target release rates. No degradation peaks were observed on HPLC chromatogram when comparing single-drug loaded ISFI to MPT ISFIs demonstrating no drug-drug interactions.

CAB release is comparable (p>0.05) when single-drug loaded and when co-formulated with either hormone. However, there is a difference (p<0.05) in the release of MPA as a single-drug ISFI and when co-formulated with CAB. MPA may be trapped in the depot until bulk degradation of the polymer begins after 30 days. This phenomenon can be explained due to MPA's low solubility in the solvent system (1:1 w/w NMP: DMSO; 33.6±0.9 mg/mL). MPA solubility in the ISFI solvent system decreases with the addition of DMSO and therefore has a lower affinity to diffuse out with the solvent during phase inversion and a higher affinity to remain trapped in PLGA until bulk degradation begins. Alternatively, there is no difference (p>0.05) in the release of ENG as a single-drug ISFI and when co-formulated with CAB up to 60 days. The release of ENG as a single-drug ISFI increases beyond 60 days and is significantly different (p<0.05) compared to ENG release when co-formulated with CAB. After 60 days of release, PLGA degradation is the main driving force influencing drug release, which is likely the reason for the increased release rate of ENG when single-drug loaded due to its higher polymer content and lower drug loading compared to the CAB/ENG ISFI.

### Example 20

### CAB ISFI Removability, Residual CAB and Polymer Degradation

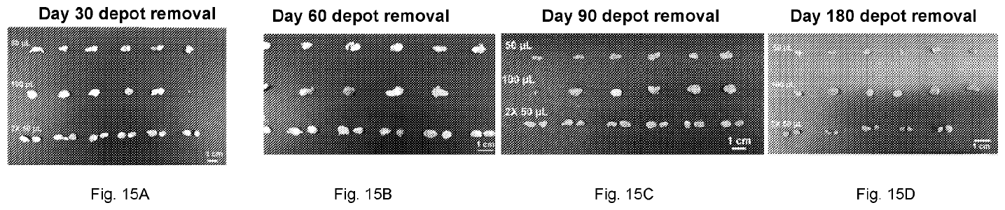

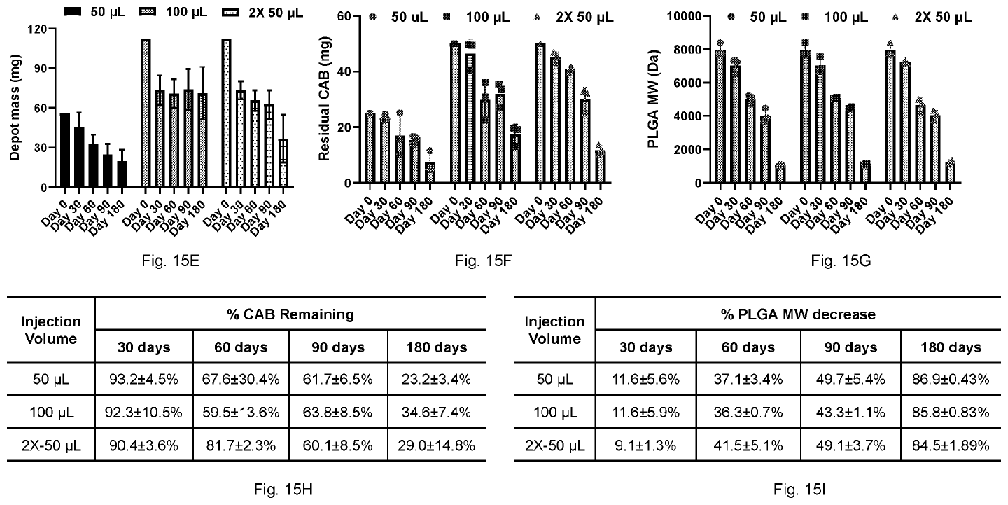

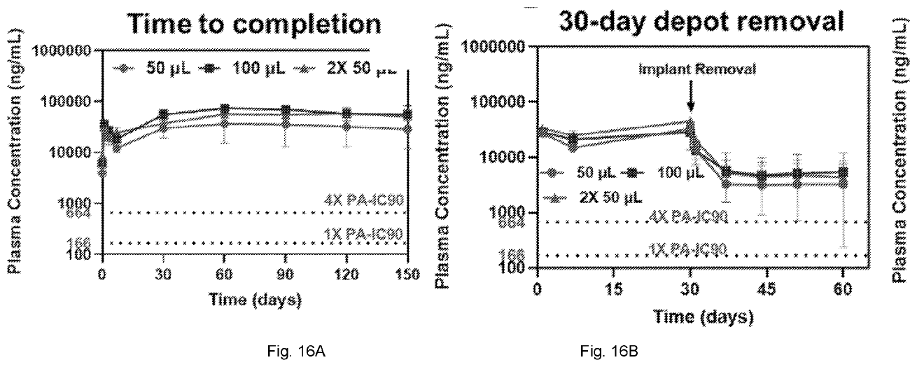

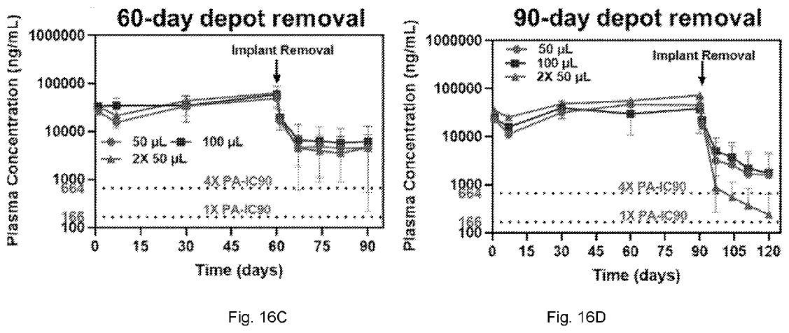

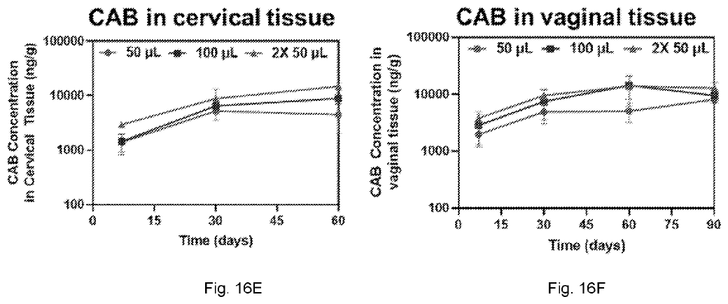

To investigate the ability to remove the implants at various time points post administrations, CAB ISFIs (500 mg/mL CAB) were administered to BALB/c mice subcutaneously at two different doses (50 μL, 100 μL, 2×50 μL) and implants were removed at d30, d60, d90, and d180 post administration. Implants were extracted to quantify residual drug by HPLC and determine the PLGA MW by GPC analysis. Plasma samples were collected for 30 days post implant removal to assess CAB tail post ISFI removal. Results show that implants were successfully retrieved at all times points (up to d180) and at d180 residual CAB was 23-30% of initial dose and PLGA MW decreased by about 85% compared to time 0 (FIG. 15H-I). Plasma PK analysis shows that CAB levels significantly decrease post ISFI removal with faster clearance of plasma CAB observed when ISFIs were removed at later timepoints (d90; FIG. 16B-D). These results demonstrate that ISFIs can successfully be removed at various timepoints post administration and drug plasma concentration is significantly reduced and cleared with short plasma tail at later time points of removal. Results also show that CAB accumulates effectively in target tissues (vaginal and rectal) at therapeutics levels for protection against HIV infection (FIG. 16E-G).

When administered at a higher dose (CAB 995 mg/kg; 100 μL injection) with the 1:4 PLGA/(NMP:DMSO) (PLGA MW 10 kDa; CAB 500 mg/gmL) suspension ISFI, plasma concentrations reached levels that were ˜50× greater than the protein-adjusted IC90 of CAB (CAB PA-IC90 166 ng/mL) for at least 30 days post-administration (FIG. 16A). These data also demonstrate minimum burst release in the first 24 h and sustained release for at least 30 days of significantly high CAB plasma levels that are well above the effective 4×PA-IC90 levels for protection against HIV acquisition.

### Example 21

### In Vivo Safety of MPT ISFI Formulations

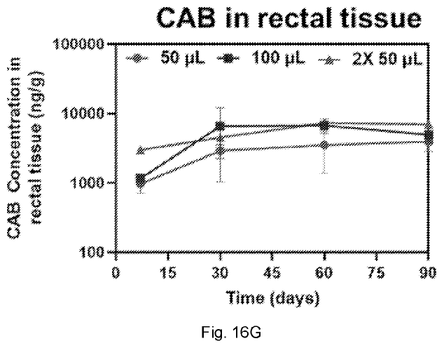

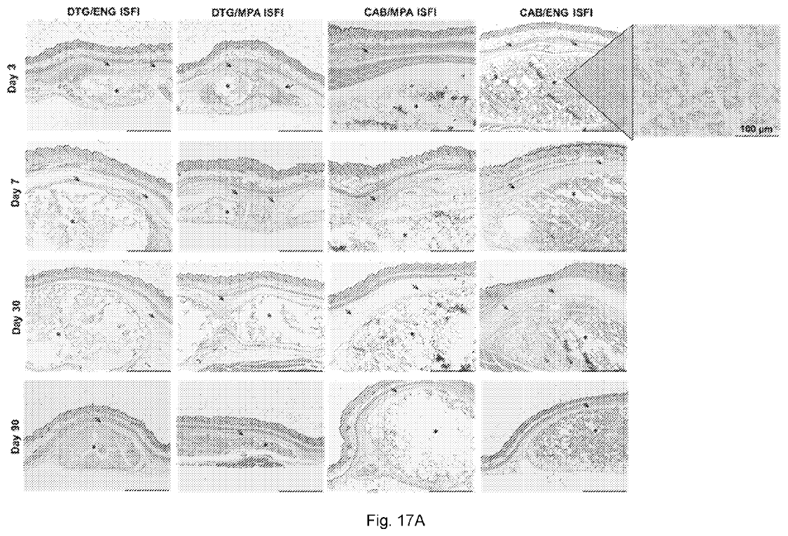

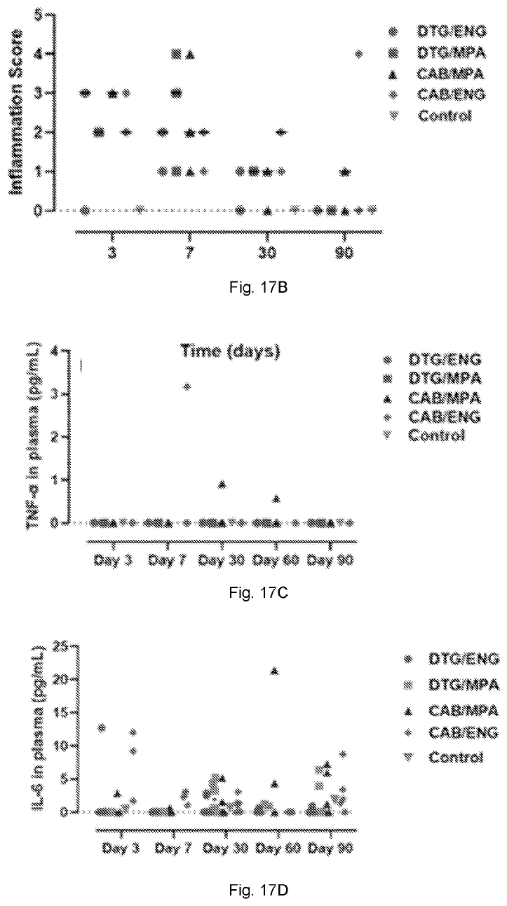

A 90-day in vivo safety study with MPT ISFI formulations (DTG/MPA, DTG/ENG, CAB/MPA, and CAB/ENG) was conducted to assess local and systemic inflammation post-injection. Results demonstrated that all ISFIs were well-tolerated, and mice did not show any signs of overt toxicity, behavioral changes, or weight loss. Histological staining analysis (H&E) of excised implant and surrounding subcutaneous tissue demonstrated that all MPT ISFI formulations exhibited mild to moderate local inflammation shown by infiltrated immune cells around the depot (FIG. 17A). The median skin microscopic inflammation scores for all MPT ISFI groups were between 2 (mild) and 3 (moderate) at day 3 and 7 due to the initial injection. Inflammation decreased by day 30 (median scores of 0 and 2) and was negligible and similar to the control group (no injection) by day 90 (median scores of 0 and 1) (FIG. 17B).

Systemic inflammation was assessed by ELISA to quantify plasma levels of TNF-α and IL-6 proinflammatory cytokines. Results showed no chronic systemic inflammation for all MPT ISFI groups. TNF-α levels were in the range of 0-3 pg/mL up to 90 days and were comparable to the control group (p>0.05) (FIG. 17C). Similarly, IL-6 levels were in the range of 0-20 pg/mL up to 90 days and were comparable to the control group (p>0.05) (FIG. 17D). Variability in inflammation scores or proinflammatory cytokines levels can be attributed to interindividual variability in mice. Ultimately, these results demonstrated that all MPT ISFIs were well-tolerated and considered safe, which, when coupled with the other data herein, demonstrates the disclosed ISFIs are well-tolerated no matter to drug included.

### Example 22

### In Vivo Pharmacokinetics of MPT ISFI Formulations

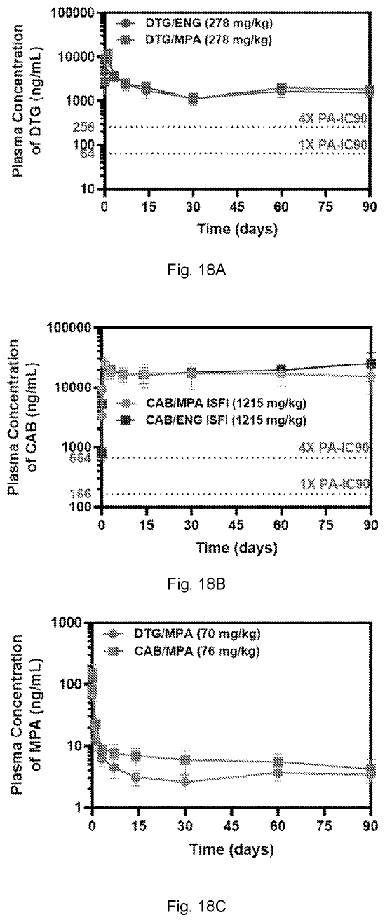

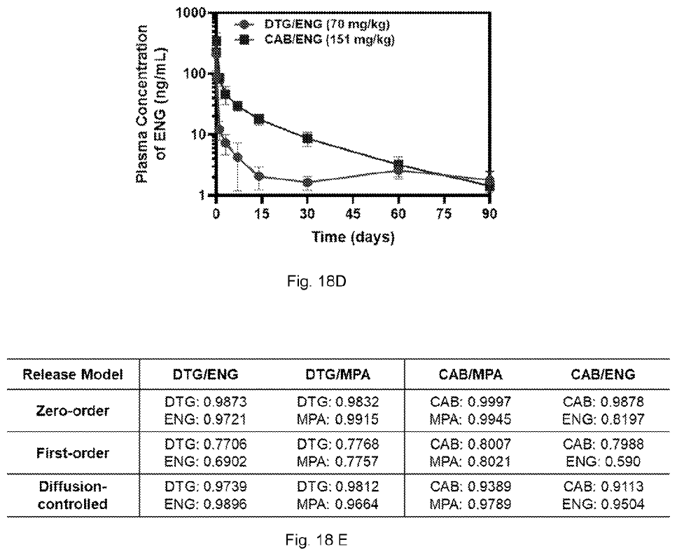

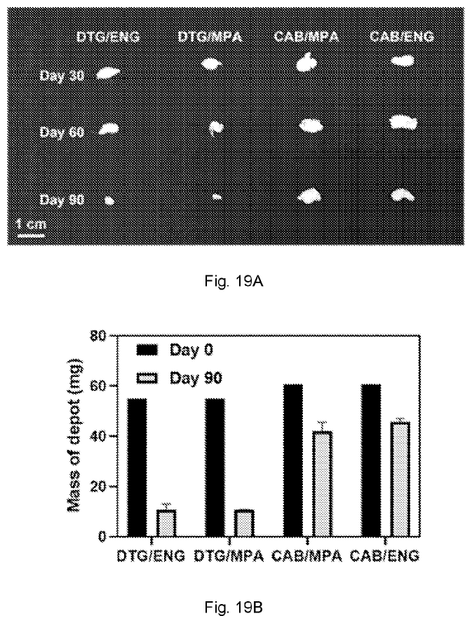

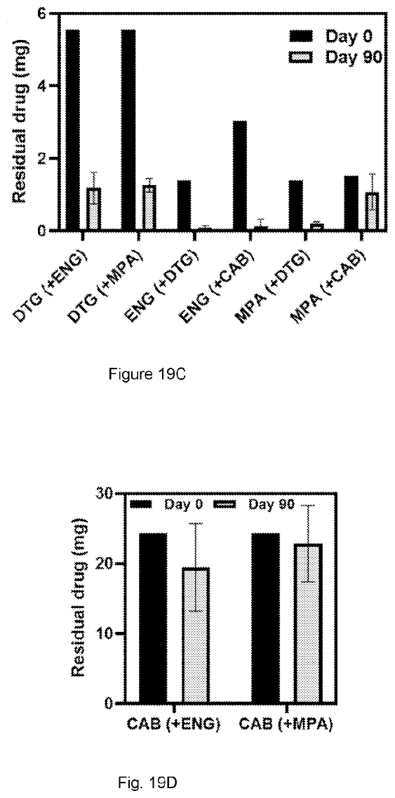

Pharmacokinetic (PK) studies were carried out in mice over 90 days to assess in vivo drug release of MPT ISFI formulations. DTG, CAB, ENG, and MPA plasma concentrations were quantified using a high-performance liquid chromatography-tandem mass spectrometry LC/MS-MS method and were plotted over 90 days (FIG. 18). Plasma concentrations of DTG and CAB in MPT ISFIs were 2-68× and 1.3-70× higher than their respective 4×PA-IC₉₀ values (DTG=256 ng/mL (Cottrell, Hadzic et al. 2013), CAB=664 ng/mL (Trezza, Ford et al. 2015), respectively, for all mice during the 90 day study and showed no difference (p>0.05) when co-formulated with either hormone (FIGS. 18A and B). Furthermore, plasma concentrations of ENG and MPA in MPT ISFIs were maintained during the 90-day study (FIGS. 18C and D).

Moreover, the release kinetics of DTG, CAB, MPA, and ENG were assessed against three mathematical models (zero-order, first-order, and diffusion-controlled (Higuchi) (Gouda, Baishya et al. 2017)). It was determined that the observed in vivo release of DTG, CAB, and MPA in mice best fit a zero-order release model and ENG release best fits a diffusion-controlled or Higuchi release model over the 90-day PK study duration (FIG. 18E).

Collectively, these results demonstrated the ability of MPT ISFI formulations to maintain plasma drug concentrations for all drugs for 90 days with ARV concentrations above the established PK benchmark (>4×PA-IC₉₀) for protection.

### Example 23

### In Vivo MPT ISFI Residual Drug Quantification and Biodegradation

The ability to easily terminate treatment is an important aspect of long-acting delivery systems, specifically for HIV prevention and contraception in the case of toxicity, adverse events, or the desire to conceive in the near future. Therefore, MPT ISFIs were removed at various time points (30, 60, and 90 days) by a small skin incision at the injection site. MPT ISFIs were successfully removed with no fibrotic tissue surrounding the depot (FIG. 19A). Depots removed at day 90 were further processed to evaluate residual drug and in vivo degradation.

DTG MPT ISFIs and CAB MPT ISFIs had approximately 82% and 28% mass loss after 90 days in vivo, respectively (FIG. 19B). CAB MPT ISFIs likely had less mass loss due to higher drug loading and lower polymer content compared to DTG MPT ISFIs. Residual drug amounts in MPT ISFIs 90 days post-administration are shown in FIGS. 19C and D. DTG MPT ISFIs had approximately 22% of DTG, 14% MPA, and 6% ENG remaining. Alternatively, CAB MPT ISFIs had approximately 85% CAB, 70% MPA, and 4% ENG remaining. Notably, CAB/MPA ISFI had a similar percent of residual drug remaining, suggesting that both drugs may reach 100% drug release simultaneously. Ultimately, all MPT ISFI formulations had drug remaining after 90 days suggesting that these formulations, especially CAB/DMPA, can release for longer. Such is expected for other drugs not tested here, but demonstrated by the example drugs herein.

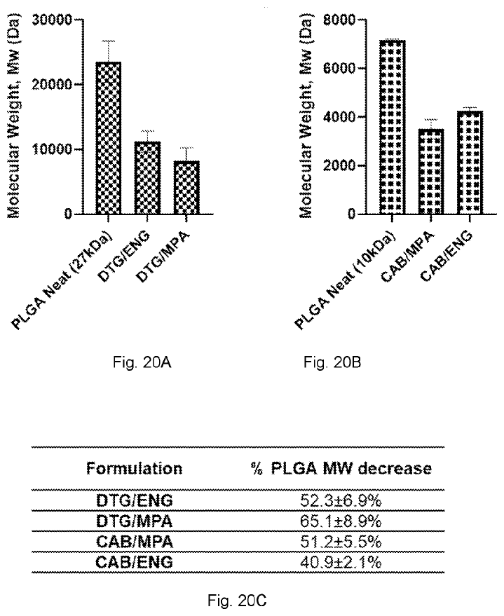

Furthermore, to assess polymer degradation, PLGA molecular weight (MW) from MPT ISFIs was quantitatively measured by GPC analysis 90 days post-injection and compared to the MW of neat PLGA (27 kDa PLGA for DTG MPT ISFIs and 10 kDa PLGA for CAB MPT ISFIs) (FIG. 20). Results showed between a 52-65% and 40-51% PLGA MW decrease in DTG MPT ISFIs and CAB MPT ISFIs, respectively. Notably, MPT ISFIs with MPA elicited a greater decrease in PLGA MW than those with ENG. This can be attributed to MPA's basic pKa increasing the rate of PLGA degradation (Holy, Dang et al. 1999, Makadia and Siegel 2011). Overall, these data demonstrate that MPT ISFIs can be easily removed if needed, depots were still intact after 90 days post-injection with residual drug remaining, indicating that these depots have potential to maintain release for longer than 90 days.

### Example 24

### ISFIs with Chemotherapeutics

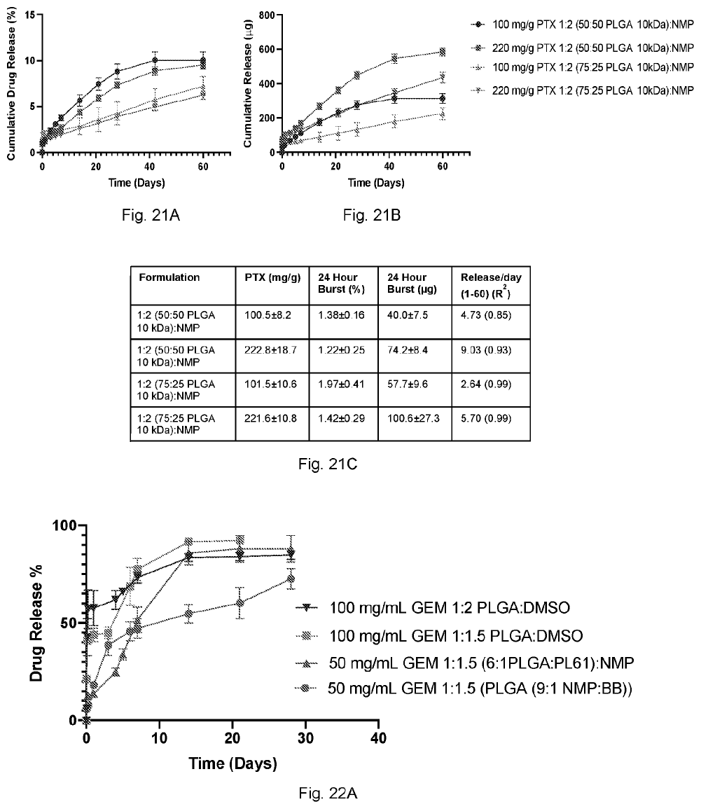

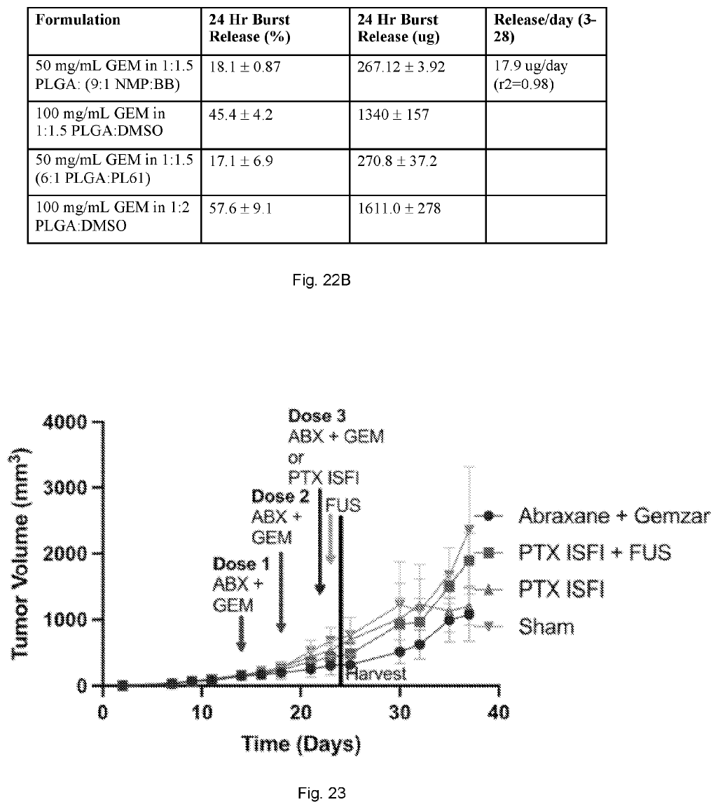

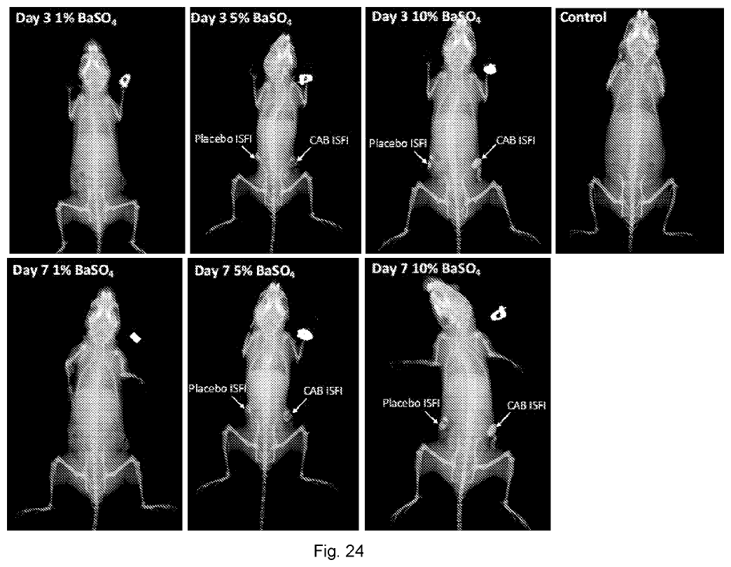

ISFI formulations were prepared with two different chemotherapeutic drugs, paclitaxel (PTX) and gemcitabine (Gem) and investigated for in vitro release kinetics (PTX and Gem ISFIs; FIGS. 21A-21C and 22A-22B) and for in vivo safety and pharmacokinetics (PTX ISFI; FIG. 23). Results showed that both PTX (MW 853.9 g/mol; LogP 3.2) and Gem (MW 263.2 g/mol; LogP −1.5) were successfully formulated in the ISFI and exhibited sustained release in vitro. Like other hydrophobic drugs (CAB, DTG, DOR), PTX exhibited a lower burst release (<2% in 24 h) and sustained release for >60 days (FIGS. 21A-21C).

### Example 25

### In Vivo Safety and PK of PTX ISFIs in Nude Mice

PTX ISFIs were administered subcutaneously (50 μL; 550 mg/kg) to KPC tumor bearing mice and plasma and tumor samples were collected longitudinally to determine the PK of PTX administered by the ISFI compared to sham and SOC (Abraxane/Gemzar combination therapy). Results showed that mice treated with PTX ISFIs had tumor size comparable to the mice that had received three doses of SOC therapy (FIG. 23). These results demonstrate that the PTX ISFIs could potentially provide a new treatment option for pancreatic cancer and other cancers that can benefit from long-acting delivery and local sustained delivery of chemotherapeutics.

### Example 26

### In Vivo X-Ray Imaging Studies of CAB+BaSO₄ ISFI

To investigate the ability to image ISFIs post administration non-invasively using full-body X-ray imaging, radiopaque placebo and CAB ISFIs containing various amounts of barium sulphate (BaSO₄; 1%, 5%, 10% w/w) were prepared and administered to BALB/c mice (n=5/group) subcutaneously (50 μL) in the flank. Each mouse received BaSO₄ containing placebo ISFI (left flank) and BaSO₄ containing CAB ISFI (right flank). X-ray images were collected at d3 and d7 post ISFI administration and compared against images collected from a control mouse (no ISFI). Results show that radiopaque CAB ISFIs containing 5% and 10% BaSO₄ were successfully prepared and were clearly visible and detectable with full-body X-ray imaging at d3 and d7 post administration (FIG. 24).

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. UNAIDS, Global HIV & AIDS statistics 2020 fact sheet. https://www.unaids.org/en/resources/fact-sheet). 2021.
2. J. M. Baeten, D. Donnell, P. Ndase, N. R. Mugo, J. D. Campbell, J. Wangisi, J. W. Tappero, E. A. Bukusi, C. R. Cohen, E. Katabira, A. Ronald, E. Tumwesigye, E. Were, K. H. Fife, J. Kiarie, C. Farquhar, G. John-Stewart, A. Kakia, J. Odoyo, A. Mucunguzi, E. Nakku-Joloba, R. Twesigye, K. Ngure, C. Apaka, H. Tamooh, F. Gabona, A. Mujugira, D. Panteleeff, K. K. Thomas, L. Kidoguchi, M. Krows, J. Revall, S. Morrison, H. Haugen, M. Emmanuel-Ogier, L. Ondrejcek, R. W. Coombs, L. Frenkel, C. Hendrix, N. N. Bumpus, D. Bangsberg, J. E. Haberer, W. S. Stevens, J. R. Lingappa, C. Celum, E. P. S. T. Partners Pr, Antiretroviral prophylaxis for HIV prevention in heterosexual men and women. N Engl J Med 367, 399-410 (2012).
3. M. C. Thigpen, P. M. Kebaabetswe, L. A. Paxton, D. K. Smith, C. E. Rose, T. M. Segolodi, F. L. Henderson, S. R. Pathak, F. A. Soud, K. L. Chillag, R. Mutanhaurwa, L. I. Chirwa, M. Kasonde, D. Abebe, E. Buliva, R. J. Gvetadze, S. Johnson, T. Sukalac, V. T. Thomas, C. Hart, J. A. Johnson, C. K. Malotte, C. W. Hendrix, J. T. Brooks, T. D. F. S. Group, Antiretroviral preexposure prophylaxis for heterosexual HIV transmission in Botswana. N Engl J Med 367, 423-434 (2012).
4. FDA, FDA Approves First Injectable Treatment for HIV Pre-Exposure Prevention. (2021) https://www.fda.gov/news-events/press-announcements/fda-approves-first-injectable-treatment-hiv-pre-exposure-prevention).
5. M. A. Marzinke, B. Grinsztejn, J. M. Fogel, E. Piwowar-Manning, M. Li, L. Weng, M. McCauley, V. Cummings, S. Ahmed, C. D. Haines, L. R. Bushman, C. Petropoulos, D. Persaud, A. Adeyeye, R. Kofron, A. Rinehart, M. St Clair, J. F. Rooney, D. Pryluka, L. Coelho, A. Gaur, K. Middelkoop, N. Phanuphak, M. S. Cohen, C. W. Hendrix, P. Anderson, B. Hanscom, D. Donnell, R. J. Landovitz, S. H. Eshleman, Characterization of Human Immunodeficiency Virus (HIV) Infection in Cisgender Men and Transgender Women Who Have Sex With Men Receiving Injectable Cabotegravir for HIV Prevention: HPTN 083. J Infect Dis 224, 1581-1592 (2021).
6. R. J. Landovitz, D. Donnell, M. E. Clement, B. Hanscom, L. Cottle, L. Coelho, R. Cabello, S. Chariyalertsak, E. F. Dunne, I Frank, J. A. Gallardo-Cartagena, A. H. Gaur, P. Gonzales, H. V. Tran, J. C. Hinojosa, E. G. Kallas, C. F. Kelley, M. H. Losso, J. V. Madruga, K. Middelkoop, N. Phanuphak, B. Santos, O. Sued, J. Valencia Huamani, E. T. Overton, S. Swaminathan, C. Del Rio, R. M. Gulick, P. Richardson, P. Sullivan, E. Piwowar-Manning, M. Marzinke, C. Hendrix, M. Li, Z. Wang, J. Marrazzo, E. Daar, A. Asmelash, T. T. Brown, P. Anderson, S. H. Eshleman, M. Bryan, C. Blanchette, J. Lucas, C. Psaros, S. Safren, J. Sugarman, H. Scott, J. J. Eron, S. D. Fields, N. D. Sista, K. Gomez-Feliciano, A. Jennings, R. M. Kofron, T. H. Holtz, K. Shin, J. F. Rooney, K. Y. Smith, W. Spreen, D. Margolis, A. Rinehart, A. Adeyeye, M. S. Cohen, M. McCauley, B. Grinsztejn, H. S. Team, Cabotegravir for HIV Prevention in Cisgender Men and Transgender Women. N Engl J Med 385, 595-608 (2021).
7. R. Landovitz, in Conference on Retroviruses and Opportunistic Infections, v. A. Abstracts of the C onference on Retroviruses and Opportunistic Infections, Ed. (Virtual, 2021).
8. C. Trezza, S. L. Ford, W. Spreen, R. Pan, S. Piscitelli, Formulation and pharmacology of long-acting cabotegravir. Curr Opin HIV AIDS 10, 239-245 (2015).
9. R. J. Landovitz, S. Li, J. J. Eron, Jr., B. Grinsztejn, H. Dawood, A. Y. Liu, M. Magnus, M. C. Hosseinipour, R. Panchia, L. Cottle, G. Chau, P. Richardson, M. A. Marzinke, S. H. Eshleman, R. Kofron, A. Adeyeye, D. Burns, A. R. Rinehart, D. Margolis, M. S. Cohen, M. McCauley, C. W. Hendrix, Tail-phase safety, tolerability, and pharmacokinetics of long-acting injectable cabotegravir in HIV-uninfected adults: a secondary analysis of the HPTN 077 trial. Lancet HIV 7, e472-e481 (2020).
10. S. R. Benhabbour, M. Kovarova, C. Jones, D. J. Copeland, R. Shrivastava, M. D. Swanson, C. Sykes, P. T. Ho, M. L. Cottrell, A. Sridharan, S. M. Fix, O. Thayer, J. M. Long, D. J. Hazuda, P. A. Dayton, R. J. Mumper, A. D. M. Kashuba, J. Victor Garcia, Ultra-long-acting tunable biodegradable and removable controlled release implants for drug delivery. Nat Commun 10, 4324 (2019).
11. M. Kovarova, S. R. Benhabbour, I. Massud, R. A. Spagnuolo, B. Skinner, C. E. Baker, C. Sykes, K. R. Mollan, A. D. M. Kashuba, J. G. Garcia-Lerma, R. J. Mumper, J. V. Garcia, Ultra-long-acting removable drug delivery system for HIV treatment and prevention. Nat Commun 9, 4156 (2018).
12. K. Al-Tahami, J. Singh, Smart polymer based delivery systems for peptides and proteins. Recent Pat Drug Deliv Formul 1, 65-71 (2007).
13. R. E. Eliaz, J. Kost, Characterization of a polymeric PLGA-injectable implant delivery system for the controlled release of proteins. J Biomed Mater Res 50, 388-396 (2000).
14. M. Parent, C. Nouvel, M. Koerber, A. Sapin, P. Maincent, A. Boudier, PLGA in situ implants formed by phase inversion: critical physicochemical parameters to modulate drug release. J Control Release 172, 292-304 (2013).
15. P. Agarwal, I. D. Rupenthal, Injectable implants for the sustained release of protein and peptide drugs. Drug Discov Today 18, 337-349 (2013).
16. R. R. Thakur, H. L. McMillan, D. S. Jones, Solvent induced phase inversion-based in situ forming controlled release drug delivery implants. J Control Release 176, 8-23 (2014).
17. R. B. Patel, A. N. Carlson, L. Solorio, A. A. Exner, Characterization of formulation parameters affecting low molecular weight drug release from in situ forming drug delivery systems. J Biomed Mater Res A 94, 476-484 (2010).
18. G. Tiwari, R. Tiwari, A. K. Rai, Cyclodextrins in delivery systems: Applications. J Pharm Bioallied Sci 2, 72-79 (2010).
19. B. D. LaMarca, D. L. Chandler, L. Grubbs, J. Bain, G. R. McLemore, Jr., J. P. Granger, M. J. Ryan, Role of sex steroids in modulating tumor necrosis factor alpha-induced changes in vascular function and blood pressure. Am J Hypertens 20, 1216-1221 (2007).
20. C. Hernandez, N. Gawlik, M. Goss, H. Zhou, S. Jeganathan, D. Gilbert, A. A. Exner, Macroporous acrylamide phantoms improve prediction of in vivo performance of in situ forming implants. J Control Release 243, 225-231 (2016).
21. C. Manaspon, C. Hernandez, P. Nittayacham, S. Jeganathan, N. Nasongkla, A. A. Exner, Increasing Distribution of Drugs Released from In Situ Forming PLGA Implants Using Therapeutic Ultrasound. Ann Biomed Eng 45, 2879-2887 (2017).
22. W. Spreen, A. Lowry, R. Pal, Y. Yueh, S. Ford, N. Richardson-Harman, J. V. Turpin, F., J. Cummins, in Conference on Retroviruses and Opportunistic Infections (CROI). (Seattle, Washington, 2015).
23. FDA, Center for Drug Evaluation and Research Integrated Review: Application Number 2128870rig1s000 (2021)). 2022.
24. C. D. Andrews, W. R. Spreen, H. Mohri, L. Moss, S. Ford, A. Gettie, K. Russell-Lodrigue, R. P. Bohm, C. Cheng-Mayer, Z. Hong, M. Markowitz, D. D. Ho, Long-acting integrase inhibitor protects macaques from intrarectal simian/human immunodeficiency virus. Science 343, 1151-1154 (2014).
25. D. Karunakaran, S. M. Simpson, J. T. Su, E. Bryndza-Tfaily, T. J. Hope, R. Veazey, G. Dobek, J. Qiu, D. Watrous, S. Sung, J. E. Chacon, P. F. Kiser, Design and Testing of a Cabotegravir Implant for HIV Prevention. J Control Release 330, 658-668 (2021).
26. T. Zhou, H. Su, P. Dash, Z. Lin, B. L. Dyavar Shetty, T. Kocher, A. Szlachetka, B. Lamberty, H. S. Fox, L. Poluektova, S. Gorantla, J. McMillan, N. Gautam, R. L. Mosley, Y. Alnouti, B. Edagwa, H. E. Gendelman, Creation of a nanoformulated cabotegravir prodrug with improved antiretroviral profiles. Biomaterials 151, 53-65 (2018).
27. T. A. Kulkami, A. N. Bade, B. Sillman, B. L. D. Shetty, M. S. Wojtkiewicz, N. Gautam, J. R. Hilaire, S. Sravanam, A. Szlachetka, B. G. Lamberty, B. M. Morsey, H. S. Fox, Y. Alnouti, J. M. McMillan, R. L. Mosley, J. Meza, P. L. Domanico, T. Y. Yue, G. Moore, B. J. Edagwa, H. E. Gendelman, A year-long extended release nanoformulated cabotegravir prodrug. Nat Mater 19, 910-920 (2020).
28. J. Radzio, W. Spreen, Y. L. Yueh, J. Mitchell, L. Jenkins, J. G. Garcia-Lerma, W. Heneine, The long-acting integrase inhibitor GSK744 protects macaques from repeated intravaginal SHIV challenge. Sci Transl Med 7, 270ra275 (2015).
29. Shaik J S, Weld E D, Edick S, Fuchs E, Riddler S, Marzinke M A, D'Amico R, Bakshi K, Lou Y, Hendrix C, Han K, Ford S L, Margolis D, Spreen W, Patel P., Multicompartmental pharmacokinetic evaluation of long-acting cabotegravir in healthy adults for HIV preexposure prophylaxis. Br J Clin Pharmacol July 8, (2021).
30. C. D. Andrews, Y. L. Yueh, W. R. Spreen, L. St Bernard, M. Boente-Carrera, K. Rodriguez, A. Gettie, K. Russell-Lodrigue, J. Blanchard, S. Ford, H. Mohri, C. Cheng-Mayer, Z. Hong, D. D. Ho, M. Markowitz, A long-acting integrase inhibitor protects female macaques from repeated high-dose intravaginal SHIV challenge. Sci Transl Med 7, 270ra274 (2015).
31. M. Marzinke, B. Grinsztejn, J. M. Fogel, E. Piwowar-Manning, B. Hanscom, E. Coelho, M. S. Cohen, A. Rinehart, J. Rooney, A. Adeyeye, P. Anderson, M. McCauley, R. J. Landovitz, S. H. Eshleman, in Conference on Retroviruses and Opportunisitic Infections. (Virtual, 2021).
32. R. J. Landovitz, D. Donnell, H. V. Tran, E. G. Kallas, M. Magnus, M. Marzinke, S. H. Eshleman, B. Hanscom, A. Adeyeye, J. Rooney, A. Rinehart, M. S. Cohen, M. McCauley, B. Grinsztejn, in Conference on Retroviruses and Opportunistic Infections. (Virtual, 2022).
33. CDC, Preexposure Prophylaxis for the Prevention of HIV Infection in the United States—2021 Update. (2021) https://www.cdc.gov/hiv/pdf/risk/prep/cdc-hiv-prep-guidelines-2021.pdf). 2022.
34. S. C. Gad, C. B. Spainhour, C. Shoemake, D. R. Pallman, A. Stricker-Krongrad, P. A. Downing, R. E. Seals, L. A. Eagle, K. Polhamus, J. Daly, Tolerable Levels of Nonclinical Vehicles and Formulations Used in Studies by Multiple Routes in Multiple Species With Notes on Methods to Improve Utility. Int J Toxicol 35, 95-178 (2016).
35. Gaylord Chemical Company LLC, DMSO Health & Safety. (2021) https://www.gaylordchemical.com/environmental-health-safety/dmso-health-safety/). 2021.
36. E. E. Tolley, S. Li, S. Z. Zangeneh, M. Atujuna, P. Musara, J. Justman, S. Pathak, L. G. Bekker, S. Swaminathan, J. Stanton, J. Farrior, N. Sista, Acceptability of a long-acting injectable HIV prevention product among US and African women: findings from a phase 2 clinical Trial (HPTN 076). J Int AIDS Soc 22, e25408 (2019).
37. A. van der Straten, K. Agot, K. Ahmed, R. Weinrib, E. N. Browne, K. Manenzhe, F. Owino, J. Schwartz, A. Minnis, T. S. Team, The Tablets, Ring, Injections as Options (TRIO) study: what young African women chose and used for future HIV and pregnancy prevention. J Int AIDS Soc 21, e25094 (2018).
38. L. Greenspan, Humidity Fixed Points of Binary Saturated Aqueous Solutions. Journal of Research of the National Bureau of Standards—A. Physics and Chemistry 81A, 89-96 (1997).
39. S. A. Vishwanathan, C. Zhao, R. Luthra, G. K. Khalil, M. M. Morris, C. Dinh, M. J. Gary, J. Mitchell, W. R. Spreen, L. E. Pereira, W. Heneine, J. G. Garcia-Lerma, J. M. McNicholl, Sexually transmitted infections and depot medroxyprogesterone acetate do not impact protection from simian HIV acquisition by long-acting cabotegravir in macaques. AIDS 36, 169-176 (2022).
40. Garcia-Lerma J G, Cong M, Mitchell J, et al, Intermittent prophylaxis with oral truvada protects macaques form rectal SHIV infection. Sci Transl Med 2, 14ra14 (2010).
41. M. A. Farage, H. I. Maibach, K. E. Andersen, J. M. Lachapelle, P. Kern, C. Ryan, J. Ely, A. Kanti, Historical perspective on the use of visual grading scales in evaluating skin irritation and sensitization. Contact Dermatitis 65, 65-75 (2011).
42. J. A. Yanez, C. M. Remsberg, C. L. Sayre, M. L. Forrest, N. M. Davies, Flip-flop pharmacokinetics—delivering a reversal of disposition: challenges and opportunities during drug development. Ther Deliv 2, 643-672 (2011).
43. R. Gouda, H. Baishya, Z. Qing, Application of Mathematical Models in Drug Release Kinetics of Carbidopa and Levodopa ER Tablets. Journal for Developing Drugs 6, 1-8 (2017).

It will be understood that various details of the presently disclosed subject matter may be changed without departing from the scope of the presently disclosed subject matter. Furthermore, the foregoing description is for the purpose of illustration only, and not for the purpose of limitation.

1. A stable polymer-based injectable suspension, the stable polymer-based injectable suspension comprising:
a polymer or a combination of polymers and stabilizer;
a solvent or a combination of solvents; and
a drug in a suspension.

2. The stable polymer-based injectable suspension of claim 1, wherein the drug is in the suspension at a concentration beyond a saturation concentration in a placebo formulation, wherein the placebo formulation comprises the polymer and the solvent.

3. The stable polymer-based injectable suspension of claim 1, wherein the stable polymer-based injectable suspension is injectable into a subject, wherein the stable polymer-based injectable suspension forms a biodegradable in-situ forming implant (ISFI) when injected into a subject.

4. The stable polymer-based injectable suspension of claim 1, wherein the stable polymer-based injectable suspension comprises one or more hydrophobic molecules or components, or a combination of hydrophobic and hydrophilic molecules.

5. The stable polymer-based injectable suspension of claim 1, wherein the ratio of polymer:solvent in the suspension ranges from about 1:1 to about 1:6.

6. The stable polymer-based injectable suspension of claim 1, wherein the ratio of polymer:drug in the suspension ranges from about 1:1 to about 1:6.

7. The stable polymer-based injectable suspension of claim 1, wherein the polymer is a biodegradable polymer selected from the group consisting of polyesters e.g. poly-lactic-co-glycolic acid (PLGA), poly-lactic acid (PLA), polyglycolic acid (PGA); polycaprolactone (PCL); Poly hydroxyl butyrate (PHB); polyethylene glycol (PEG); sucrose acetate isobutyrate (SAIB); polyamides; polyanhydrides; polyphosphazenes; polyacrylates; polyorthoesters; polyalkylcyanoacrylates; polyurethanes; poly(ester amides); poly(ester urea); poly(phosphoesters); polysaccharides; hyaluronic acid; chitosan; alginate; collagen; arginine; albumin; dextran; gelatin; agarose; carrageenan; biomimetic and bio-inspired polymers or combinations thereof.

8. The stable polymer-based injectable suspension of claim 1, wherein the molecular weight (MW) of the polymer ranges from about 5 kDa to about 100 kDa.

9. The stable polymer-based injectable suspension of claim 1, wherein the solvent is a water-miscible biocompatible solvent selected from the group consisting of N-methyl-2-pyrrolidone (NMP), dimethyl sulfoxide (DMSO), benzyl benzoate (BB), triacetin (TA) and combinations thereof.

10. The stable polymer-based injectable suspension of claim 1, wherein the solvent comprises a mixture of NMP and DMSO at a ratio of about 1:1 v/v to about 1:9 v/v.

11. The stable polymer-based injectable suspension of claim 1, wherein the stable polymer-based injectable suspension comprises one or more drugs, biologics, active agents, contrast agents and/or therapeutic compounds.

12. (canceled)

13. (canceled)

14. The stable polymer-based injectable suspension of claim 1, wherein the suspension comprises the polymer PLGA and a NMP and DMSO solvent mixture at a ratio of about 1:2 to about 1:6, with a drug at a concentration of about 200 mg/g to about 600 mg/g.

15. The stable polymer-based injectable suspension of claim 1, wherein the suspension has a high drug loading capacity up to about 600 mg/mL.

16. The stable polymer-based injectable suspension of claim 1, wherein the suspension is configured to accommodate one or more drugs at concentrations ranging from about 5 wt % to about 85 wt % which is translatable to a human dose required to achieve therapeutic effect.

17. The stable polymer-based injectable suspension of claim 1, wherein the suspension is configured to provide ultra-long-acting drug release of about 90 days or more.

18. (canceled)

19. (canceled)

20. The stable polymer-based injectable suspension of claim 1, wherein the suspension is removable from a subject after injection.

21. (canceled)

22. (canceled)

23. A biodegradable in-situ forming implant (ISFI) made from a stable polymer-based injectable suspension of claim 1.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A method of administering a drug, active agent, contrast agent and/or therapeutic compound to a subject, the method comprising providing a stable polymer-based injectable suspension of claim 1, and administering the same to a subject in need of receiving a drug, biologic, active agent, contrast agent and/or therapeutic compound.

52. The method of claim 51, further comprising loading the stable polymer-based injectable suspension with one or more drugs, active agents, contrast agent and/or therapeutic compounds prior to administration to the subject, optionally wherein the one or more drugs, active agents and/or therapeutic compounds comprises an antiviral, antibacterial, antifungal, contraceptive, prophylactic, anti-inflammatory, anticancer, analgesic, hormone, steroid, opioid and combinations thereof.

53. The method of claim 51, wherein the stable polymer-based injectable suspension a is administered via injection.

54. The method of claim 51, wherein the stable polymer-based injectable suspension is configured to be removable from the subject if required to terminate the treatment.

55. (canceled)

56. (canceled)

57. (canceled)

58. (canceled)

59. (canceled)
